SEQUENCE LISTING (115 sequences)

SEQ ID NO: 1 - DNA, 297 bp, Artificial Sequence
  source: /note=“Description of Artificial Sequence: Synthetic polynucleotide”
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaa

SEQ ID NO: 2 - protein, 98 aa, Artificial Sequence
  source: /note=“Description of Artificial Sequence: Synthetic polypeptide”
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 3 - DNA, 405 bp, Artificial Sequence
  source: /note=“Description of Artificial Sequence: Synthetic polynucleotide”
atggctcagtcactggctctgagcctccttatcctggttctggcctttggcatccccaggacccaaggcagtgatggaggggctcaggactgttgcctcaagtacagccaaaggaagattcccgccaaggttgtccgcagctaccggaagcaggaaccaagcttaggctgctccatcccagctatcctgttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggactgagcggtcacagacccctaaagggccatag

SEQ ID NO: 4 - protein, 134 aa, Artificial Sequence
  source: /note=“Description of Artificial Sequence: Synthetic polypeptide”
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 5 - DNA, 300 bp, Artificial Sequence
  source: /note=“Description of Artificial Sequence: Synthetic polynucleotide”
atgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataa

SEQ ID NO: 6 - protein, 99 aa, Artificial Sequence
  source: /note=“Description of Artificial Sequence: Synthetic polypeptide”
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 7 - DNA, 324 bp, Artificial Sequence
  source: /note=“Description of Artificial Sequence: Synthetic polynucleotide”
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactga

SEQ ID NO: 8 - protein, 107 aa, Artificial Sequence
  source: /note=“Description of Artificial Sequence: Synthetic polypeptide”
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 9 - DNA, 336 bp, Artificial Sequence
  source: /note=“Description of Artificial Sequence: Synthetic polynucleotide”
atgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 10 - protein, 111 aa, Artificial Sequence
  source: /note=“Description of Artificial Sequence: Synthetic polypeptide”
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 11 - DNA, 1119 bp, Artificial Sequence
  source: /note=“Description of Artificial Sequence: Synthetic polynucleotide”
atgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaa

SEQ ID NO: 12 - protein, 372 aa, Artificial Sequence [sequence omitted]

SEQ ID NO: 13 - DNA, 1116 bp, Artificial Sequence
  source: /note=“Description of Artificial Sequence: Synthetic polynucleotide”
atgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctcccca

SEQ ID NO: 14 - protein, 372 aa, Artificial Sequence
  source: /note=“Description of Artificial Sequence: Synthetic polypeptide”
MKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 15 - DNA, 1083 bp, Artificial Sequence
  source: /note=“Description of Artificial Sequence: Synthetic polynucleotide”
atggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaa

SEQ ID NO: 16 - protein, 360 aa, Artificial Sequence
  source: /note=“Description of Artificial Sequence: Synthetic polypeptide”
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 17 - DNA, 1178 bp, Artificial Sequence
  source: /note=“Description of Artificial Sequence: Synthetic polynucleotide”
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 18 - DNA, 5228 bp, Artificial Sequence
  source: /note=“Description of Artificial Sequence: Synthetic polynucleotide”
tctagagctgaccaaaagagcaccaaaggcgccctgaccttcagcccctacctgcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgataatacgactcactatagggagacccaagctggaattcggcggccgccaccatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttaatgcataacttcgtataatgtatgctatacgaagttatgttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagtgtaacgccaccatgaccgagtacaagcctaccgtgaggctggccacccgggacgacgtgcccagagccgtgaggacactggccgccgccttcgccgactaccccgccacccggcacaccgtggaccccgaccggcacatcgagcgggtgaccgagctgcaggaactgttcctgaccagagtgggcctggatatcggcaaagtgtgggtggccgacgacggagccgccgtggccgtgtggaccacccccgagtccgtggaggccggagccgtgtttgccgagatcggccccaggatggccgagctgtccggcagcaggctggccgcccagcagcagatggaaggcctgctggcccctcaccggcccaaagagcccgcctggttcctggccaccgtgggcgtgagccccgaccaccagggcaagggcctgggcagcgccgtggtgctgccaggcgtggaagccgccgagagggccggagtgcccgccttcctggaaaccagcgcccccaggaacctgcccttctacgagcggctgggctttaccgtgaccgccgacgtggaggtgccagagggccccaggacctggtgcatgacccggaagccaggcgcctgagaaaagcttataacttcgtataatgtatgctatacgaagttataacttgtttattgcagcttataatggttacaaataaagcaatagcatcacaaatttcacaaataaagcatttttttcactgcattctagttgtggtttgtccaaactcatcaatgtatcttatcatgtctgtgcggtgggctctatggcttctgaggcggaaagaaccagctggggctctagggggtatcccctctaga

SEQ ID NO: 19 - DNA, 1211 bp, Artificial Sequence
  source: /note=“Description of Artificial Sequence: Synthetic polynucleotide”
ggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 20 - DNA, 500 bp, Artificial Sequence
  source: /note=“Description of Artificial Sequence: Synthetic polynucleotide”
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctacccctcccctagtcaggaagttcccccccgccccgcagctcgcgtcatgcaggacgtgacaaatggaagtagcacgtctcactagtctcgtgcaaatggacagcaccgctgagcaatggaagcgggtaggcccttggggcagcggccaatagcagctttgctccttcgctttctgggctcagaggctgggaaggggtgggtccgggggcgggctcaggggcgggctcaggggcggggcgggcgcccgaaggtcctccggaggcccggcattccgcacgcttcaaaagcgcacgtctgccgcgctgttctcttcttcctcatctccgggcctttcg

SEQ ID NO: 21 - DNA, 508 bp, Artificial Sequence
  source: /note=“Description of Artificial Sequence: Synthetic polynucleotide”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 22 - protein, 254 aa, Artificial Sequence
  source: /note=“Description of Artificial Sequence: Synthetic polypeptide”
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 23 - DNA, 765 bp, Artificial Sequence
  source: /note=“Description of Artificial Sequence: Synthetic polynucleotide”
atgtggcagctgctgctgcctacagctctcctgctgctggtgtccgccggcatgagaaccgaggatctgcctaaggccgtggtgttcctggaaccccagtggtacagagtgctggaaaaggacagcgtgaccctgaagtgccagggcgcctacagccccgaggacaatagcacccagtggttccacaacgagagcctgatcagcagccaggccagcagctacttcatcgacgccgccaccgtggacgacagcggcgagtatagatgccagaccaacctgagcaccctgagcgaccccgtgcagctggaagtgcacatcggatggctgctgctgcaggcccccagatgggtgttcaaagaagaggaccccatccacctgagatgccactcttggaagaacaccgccctgcacaaagtgacctacctgcagaacggcaagggcagaaagtacttccaccacaacagcgacttctacatccccaaggccaccctgaaggactccggctcctacttctgcagaggcctcgtgggcagcaagaacgtgtccagcgagacagtgaacatcaccatcacccagggcctggccgtgtctaccatcagcagctttttcccacccggctaccaggtgtccttctgcctcgtgatggtgctgctgttcgccgtggacaccggcctgtacttcagcgtgaaaacaaacatcagaagcagcacccgggactggaaggaccacaagttcaagtggcggaaggacccccaggacaagtga

SEQ ID NO: 24 - DNA, 483 bp, Artificial Sequence
  source: /note=“Description of Artificial Sequence: Synthetic polynucleotide”
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctggaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgaccggctccgagaaggacgagctgtga

SEQ ID NO: 25 - protein, 160 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 26 - DNA, 60 bp, Artificial Sequence
  source: /note=“Description of Artificial Sequence: Synthetic oligonucleotide”
cagtgcaccaactacgccctgctgaagctggccggcgacgtggagagcaaccctggccct

SEQ ID NO: 27 - DNA, 54 bp, Artificial Sequence
  source: /note=“Description of Artificial Sequence: Synthetic oligonucleotide”
gagggcagaggcagcctgctgacctgcggcgatgtggaggaaaacccaggccca

SEQ ID NO: 28 - DNA, 19 bp, Artificial Sequence
  source: /note=“Description of Artificial Sequence: Synthetic oligonucleotide”
taatacgactcactatagg

SEQ ID NO: 29 - DNA, 66 bp, Artificial Sequence
  source: /note=“Description of Artificial Sequence: Synthetic oligonucleotide”
ggatctggagctactaacttcagcctgctgaagcaggctggagacgtggaggagaaccctggacct

SEQ ID NO: 30 - DNA, 69 bp, Artificial Sequence
  source: /note=“Description of Artificial Sequence: Synthetic oligonucleotide”
atggactggatctggcggattctgtttctcgtgggagctgccacaggcgctcattctgctcagcctgcc

SEQ ID NO: 31 - DNA, 1152 bp, Artificial Sequence
  source: /note=“Description of Artificial Sequence: Synthetic polynucleotide”
gatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 32 - DNA, 592 bp, Artificial Sequence
  source: /note=“Description of Artificial Sequence: Synthetic polynucleotide”
cgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataaccgc

SEQ ID NO: 33 - DNA, 252 bp, Artificial Sequence
  source: /note=“Description of Artificial Sequence: Synthetic polynucleotide”
acgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 34 - protein, 41 aa, Artificial Sequence
  source: /note=“Description of Artificial Sequence: Synthetic polypeptide”
LKIQVRKAAITSYEKSDGVYTGLSTRNQETYETLKHEKPPQ

SEQ ID NO: 35 - DNA, 123 bp, Artificial Sequence
  source: /note=“Description of Artificial Sequence: Synthetic polynucleotide”
ctgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 36 - protein, 64 aa, Artificial Sequence
  source: /note=“Description of Artificial Sequence: Synthetic polypeptide”
LSNSIMYFSHFVPVFLPAKPTTTPAPRPPTPAPTIASQPLSLRPEACRPAAGGAVHTRGLDFAC

SEQ ID NO: 37 - DNA, 192 bp, Artificial Sequence
  source: /note=“Description of Artificial Sequence: Synthetic polynucleotide”
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgc

SEQ ID NO: 38 - protein, 28 aa, Artificial Sequence
  source: /note=“Description of Artificial Sequence: Synthetic peptide”
FWVLVVVGGVLACYSLLVTVAFIIFWVR

SEQ ID NO: 39 - DNA, 84 bp, Artificial Sequence
  source: /note=“Description of Artificial Sequence: Synthetic oligonucleotide”
ttttgggtgctggtggtcgtgggcggagtgctggcttgttattctctgctggtcaccgtggccttcatcatcttttgggtccga

SEQ ID NO: 40 - protein, 133 aa, Artificial Sequence
  source: /note=“Description of Artificial Sequence: Synthetic polypeptide”
LSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 41 - DNA, 399 bp, Artificial Sequence
  source: /note=“Description of Artificial Sequence: Synthetic polynucleotide”
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 42 - protein, 252 aa, Artificial Sequence
  source: /note=“Description of Artificial Sequence: Synthetic polypeptide”
AQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 43 - protein, 113 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - DNA, 1410 bp, Artificial Sequence
  source: /note=“Description of Artificial Sequence: Synthetic polynucleotide”
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcaaggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgagtgaagttcagcagatccgccgatgcccctgcttaccagcagggccagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 45 - protein, 153 aa, Artificial Sequence
  source: /note=“Description of Artificial Sequence: Synthetic polypeptide”
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 46 - protein, 398 aa, Artificial Sequence
  source: /note=“Description of Artificial Sequence: Synthetic polypeptide”
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSAAALFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ

SEQ ID NO: 47 - protein, 259 aa, Artificial Sequence
  source: /note=“Description of Artificial Sequence: Synthetic polypeptide”
AATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 48 - DNA, 1197 bp, Artificial Sequence
  source: /note=“Description of Artificial Sequence: Synthetic polynucleotide”
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctgctgctgctctgttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccagtaa

SEQ ID NO: 49 - protein, 54 aa, Artificial Sequence
  source: /note=“Description of Artificial Sequence: Synthetic polypeptide”
KPTTTPAPRPPTPAPTIASQPLSLRPEACRPAAGGAVHTRGLDFACFWVLVVVG

SEQ ID NO: 50 - protein, 20 aa, Artificial Sequence
  source: /note=“Description of Artificial Sequence: Synthetic peptide”
GVLACYSLLVTVAFIIFWVR

SEQ ID NO: 51 - protein, 41 aa, Artificial Sequence
  source: /note=“Description of Artificial Sequence: Synthetic polypeptide”
RSKRSRLLHSDYMNMTPRRPGPTRKHYQPYAPPRDFAAYRS

SEQ ID NO: 52 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 53 - DNA, 339 bp, Artificial Sequence
  source: /note=“Description of Artificial Sequence: Synthetic polynucleotide”
gtgaagtttagcagatctgccgacgcccctgcctaccagcagggacagaatcagctgtacaacgagctgaacctgggcagacgggaagagtacgacgtgctggataagagaagaggcagagatcccgagatgggcggcaagccccagagaagaaagaatccccaggaaggcctgtataacgaactgcagaaagacaagatggccgaggcctacagcgagatcggcatgaagggcgagagaagaagaggcaagggccacgatggactgtaccagggactgagcacagccaccaaggatacctacgatgccctgcacatgcaggccctgcctccaagataa

SEQ ID NO: 54 - DNA, 339 bp, Artificial Sequence
  source: /note=“Description of Artificial Sequence: Synthetic polynucleotide”
gtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 55-61 - skipped (7 entries)

SEQ ID NO: 62 - DNA, 813 bp, Artificial Sequence
  source: /note=“Description of Artificial Sequence: Synthetic polynucleotide”
atggactggatctggcggattctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatct

SEQ ID NO: 63 - protein, 271 aa, Artificial Sequence
  source: /note=“Description of Artificial Sequence: Synthetic polypeptide”
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 64 - DNA, 807 bp, Artificial Sequence
  source: /note=“Description of Artificial Sequence: Synthetic polynucleotide”
atggactggatctggcgcatcctcttcctcgtcggcgctgctaccggcgctcattcggcccagccggccatggcgcaagtaaaactccaagaatctggggcggagctggtgaaaccgggggcgtctgtgaagatgagctgtaaagcatcaggctacaccttcacctcctataatatgcactgggtgaaacaaacacccggacagggcctcgaatggattggtgccatctatcctggaaatggtgatacctcatataatcagaagtttaagggcaaggctacgcttactgcggataaaagctcttccactgcttacatgcaactgagcagtctcacttcagaggactcagccgattattattgtgcccgcagcaactactatggtagttcatactggtttttcgacgtttgggggcaaggtaccaccgtcacggtttcttctggtgggggcggaagcgggggtggaggatctgggggcggtggttcagacattgaactcacccagagccctactattctgagcgcgtctccaggtgaaaaagttacgatgacgtgcagagcatcaagtagtgtgaattatatggattggtatcaaaagaagccaggctcatccccaaaaccgtggatctatgcaactagcaacctcgcgtcaggggtgccagcaaggttttccggaagtggttctggcacatcttatagtctcaccatttcccgagtggaggctgaggatgcggccacttattactgccagcaatggtcattcaatcccccaacatttggtggcggaacaaaactcgaaattaaacgg

SEQ ID NO: 65 - protein, 269 aa, Artificial Sequence
  source: /note=“Description of Artificial Sequence: Synthetic polypeptide”
MDWIWRILFLVGAATGAHSAQPAMAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKR

SEQ ID NO: 66 - DNA, 795 bp, Artificial Sequence
  source: /note=“Description of Artificial Sequence: Synthetic polynucleotide”
atggactggatctggcgcatcctcttcctcgtcggcgctgctaccggcgctcattcggcccagccggccgacattcaaatgactcagtccccttccagcttgtcagcctcagtaggggaccgggtcacgatcacctgtcgagcgtctgagtcagtggataactacgggatttctttcatgaactggttccagcagaagcccggcaaagctcctaagctccttatatatgcagcctcaaatcaggggagcggtgttcctagtcgcttcagtggaagcggtagcggtacggactttacgttgacgataagtagccttcagccagatgactttgccacttattattgtcagcagtctaaggaagttccttggacgtttggccaaggaacgaaggtcgaaatcaaagggggagggggctcaggagggggcggcagtggtggtggaggctctcaagtccaactcgtacagtctggcgcggaggttaaaaagccgggaagctccgtgaaagtatcctgtaaggcaagcggatacacctttaccgattataacatgcactgggttaggcaggcgcccggccaaggtctggaatggatcggttatatttatccatacaacggtggtaccggctataatcagaagtttaagagtaaggctactattacagcggatgagtcaaccaatactgcatacatggagctctcctcactcaggagcgaagataccgcagtgtattactgtgcccgagggagaccagccatggactactggggtcagggtacccttgtgacagtatctagc

SEQ ID NO: 67 - protein, 265 aa, Artificial Sequence
  source: /note=“Description of Artificial Sequence: Synthetic polypeptide”
MDWIWRILFLVGAATGAHSAQPADIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 68 - DNA, 840 bp, Artificial Sequence
  source: /note=“Description of Artificial Sequence: Synthetic polynucleotide”
atggactggatctggcgcatcctcttcctcgtcggcgctgctaccggcgctcattcggcccagccggccgatatcgagctcacccaatctccaaaattcatgtccacatcagtaggagacagggtcagcgtcacctgcaaggccagtcagaatgtggatactaatgtagcgtggtatcaacaaaaaccagggcaatctcctgaaccactgcttttctcggcatcctaccgttacactggagtccctgatcgcttcacaggcagtggatctgggacagatttcactctcaccatcagcaatgtgcagtctgaagacttggcagagtatttctgtcagcaatataacagctatcctctgacgttcggtggcggcaccaagctggaaatcaaacgggctgccgcagaaggtggaggcggttcaggtggcggaggttccggcggaggtggctctggcggtggcggatcggccatggcccaggtgaagctgcagcagtcaggagggggcttggtgcaacctggaggctccatgaaactctcctgtgttgtctctggattcactttcagtaattactggatgaactgggtccgccagtctccagagaaggggcttgagtggattgcagaaattagattgaaatccaataattttggaagatattatgcggagtctgtgaaagggaggttcaccatctcaagagatgattccaaaagtagtgcctacctgcaaatgatcaacctaagagctgaagatactggcatttattactgtaccagttatggtaactacgttgggcactattttgaccactggggccaagggaccacggtcaccgtatcgagt

SEQ ID NO: 69 - protein, 280 aa, Artificial Sequence
  source: /note=“Description of Artificial Sequence: Synthetic polypeptide”
MDWIWRILFLVGAATGAHSAQPADIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAAAEGGGGSGGGGSGGGGSGGGGSAMAQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 70 - DNA, 807 bp, Artificial Sequence
  source: /note=“Description of Artificial Sequence: Synthetic polynucleotide”
atggactggatctggcggattctgtttctcgtgggagctgccacaggcgctcattctgctcagcctgccgatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaaggcggcggaggaagcggaggcggaggatctgggggcggaggctctggcggagggggatctcaggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcg

SEQ ID NO: 71 - protein, 269 aa, Artificial Sequence
  source: /note=“Description of Artificial Sequence: Synthetic polypeptide”
MDWIWRILFLVGAATGAHSAQPADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 72 - DNA, 822 bp, Artificial Sequence
  source: /note=“Description of Artificial Sequence: Synthetic polynucleotide”
atggactggatctggcggattctgtttctcgtgggagctgccacaggcgctcattctgctcagcctgccgatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaaggcggcggaggaagcggaggcggaggatctgggggcggaggctctggcggagggggatctcaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatcc

SEQ ID NO: 73 - protein, 274 aa, Artificial Sequence
  source: /note=“Description of Artificial Sequence: Synthetic polypeptide”
MDWIWRILFLVGAATGAHSAQPADVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSS

SEQ ID NO: 74 - DNA, 822 bp, Artificial Sequence
  source: /note=“Description of Artificial Sequence: Synthetic polynucleotide”
atggactggatctggcggattctgtttctcgtgggagctgccacaggcgctcattctgctcagcctgccgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagggcggcggaggaagcggaggcggaggatctgggggcggaggctctggcggagggggatctgaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagc

SEQ ID NO: 75 - protein, 274 aa, Artificial Sequence
  source: /note=“Description of Artificial Sequence: Synthetic polypeptide”
MDWIWRILFLVGAATGAHSAQPADIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKGGGGSGGGGSGGGGSGGGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSS

SEQ ID NO: 76 - DNA, 825 bp, Artificial Sequence
  source: /note=“Description of Artificial Sequence: Synthetic polynucleotide”
atggactggatctggcggattctgtttctcgtgggagctgccacaggcgctcattctgctcagcctgccgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaagtcctcagggggcgggggaagtggtgggggcggcagcggcggagggggctcaggaggaggcggatcaggcggatcagaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 77 - protein, 275 aa, Artificial Sequence
  source: /note=“Description of Artificial Sequence: Synthetic polypeptide”
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 78 - DNA, 798 bp, Artificial Sequence
  source: /note=“Description of Artificial Sequence: Synthetic polynucleotide”
atggactggatctggcggattctgtttctcgtgggagctgccacaggcgctcattctgctcagcctgccagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaaggcggcggaggaagcggaggcggaggatctgggggcggaggctctggcggagggggatctcaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatct

SEQ ID NO: 79 - DNA, 798 bp, Artificial Sequence
  source: /note=“Description of Artificial Sequence: Synthetic polynucleotide”
atggactggatctggcggattctgtttctcgtgggagctgccacaggcgctcattctgctcagcctgcccaggtgcaagtgaaagagtctggccctggactggtggccccaagccagtctctgagcatcacatgtaccgtgtccggcttcagcctgaccaactatggcgtgcactgggtccgacagcctccaggcaaaggactggaatggctgggagtgatttgggctggcggcagcaccaactacaacagcgccctgatgagccggctgagcatctccaaggacaacagcaagagccaggtgttcctgaagatgaacagcctgcagaccgacgacaccgccatgtactactgtgctagcagaggcggcaactacggctacgccctggattattggggccagggcacaagcgtgaccgtgtcatctggcggcggaggaagcggaggcggaggatctgggggcggaggctctggcggagggggatctagcatcgtgatgacccagactcctaagttcctgctggtgtctgccggcgacagagtgaccatcacctgtaaagccagccagagcgtgtccaacgacgtggcctggtatcagcagaagcctggacagagccccaagctgctgatctacagcgccagcaacagatacaccggcgtgcccgatagattcaccggctctggctacggcaccgacttcacctttaccatcagcaccgtgcaggccgaggatctggccgtgtacttctgccagcaagactacagctctctcggcggaggcaccaagctggaaatcaaa

SEQ ID NO: 80 - protein, 266 aa, Artificial Sequence
  source: /note=“Description of Artificial Sequence: Synthetic polypeptide”
MDWIWRILFLVGAATGAHSAQPASIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIKGGGGSGGGGSGGGGSGGGGSQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSS

SEQ ID NO: 81 - protein, 266 aa, Artificial Sequence
  source: /note=“Description of Artificial Sequence: Synthetic polypeptide”
MDWIWRILFLVGAATGAHSAQPAQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSSGGGGSGGGGSGGGGSGGGGSSIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIK

SEQ ID NO: 82 - protein, 61 aa, Artificial Sequence
  source: /note=“Description of Artificial Sequence: Synthetic polypeptide”
LSNSIMYFSHFVPVFLPAKPTTTPAPRPPTPAPTIASQPLSLRPEACRPAAGGAVHTRGLD

SEQ ID NO: 83 - DNA, 183 bp, Artificial Sequence
  source: /note=“Description of Artificial Sequence: Synthetic polynucleotide”
ctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggat

SEQ ID NO: 84 - protein, 135 aa, Artificial Sequence
  source: /note=“Description of Artificial Sequence: Synthetic polypeptide”
PKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - DNA, 822 bp, Artificial Sequence
  source: /note=“Description of Artificial Sequence: Synthetic polynucleotide”
atggactggatctggcggattctgtttctcgtgggagctgccacaggcgctcattctgctcagcctgccctgcccgtcctgacacagtccgcaagtgtgagtggatcacctggacaatcaattactatcagttgcacaggcacttcctcagacgtagggaggtatgattacgtgtcatggtatcaacagcatccaggcaaagctcctcagctcatgatttatgatgtgtccaacagaccgtccggagtatctaatcgcttcagtggatctaaatccggtaatactgcctccctcaccatatcagggctccaggccgaagatgaagcagactactattgcagtagttacactggttcaagtacgctttacgtttttggcacggggaccaaggtaacggtcctgggccaacccaaaggcggaggagggtccggtggcggtggcagtggtggagggggatcagaggtgcaattggttgagagcggtggtgggctggttaaacctggcgggtccctccgcttgtcttgtgccgcaagcgggtttacctttagtaatgcgtggatgagctgggtgcgacaagcacccggaaagggcctggagtgggtcggtaggattaaaagcaaaacagatggtggaacaaccgattatgcggccccagtcaagggaaggttcactatttcaagagacgattccaagaacactctttacctccaaatgaatagtttgaaaacagaggatacagcagtgtactattgcacaacggactacgacttttggagcggatattactactgggggcaaggtaccctggtcacagtttcatca

SEQ ID NO: 86 - protein, 274 aa, Artificial Sequence
  source: /note=“Description of Artificial Sequence: Synthetic polypeptide”
MDWIWRILFLVGAATGAHSAQPALPVLTQSASVSGSPGQSITISCTGTSSDVGRYDYVSWYQQHPGKAPQLMIYDVSNRPSGVSNRFSGSKSGNTASLTISGLQAEDEADYYCSSYTGSSTLYVFGTGTKVTVLGQPKGGGGSGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDYDFWSGYYYWGQGTLVTVSS

SEQ ID NO: 87 - DNA, 807 bp, Artificial Sequence
  source: /note=“Description of Artificial Sequence: Synthetic polynucleotide”
atggactggatctggcggattctgtttctcgtgggagctgccacaggcgctcattctgctcagcctgccaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctca

SEQ ID NO: 88 - protein, 269 aa, Artificial Sequence
  source: /note=“Description of Artificial Sequence: Synthetic polypeptide”
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSS

SEQ ID NO: 89 - DNA, 807 bp, Artificial Sequence
  source: /note=“Description of Artificial Sequence: Synthetic polynucleotide”
atggactggatctggcggattctgtttctcgtgggagctgccacaggcgctcattctgctcagcctgccgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtaccctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaag

SEQ ID NO: 90 - protein, 269 aa, Artificial Sequence
  source: /note=“Description of Artificial Sequence: Synthetic polypeptide”
MDWIWRILFLVGAATGAHSAQPAEVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIK

SEQ ID NO: 91-92 - skipped (2 entries)

SEQ ID NO: 93 - DNA, 840 bp, Artificial Sequence
  source: /note=“Description of Artificial Sequence: Synthetic polynucleotide”
atggactggatctggcggattctgtttctcgtgggagctgccacaggcgctcattctgctcagcctgccgacatcgtgatgacacagagcccttctagcctggccgtgtccgtgggagagaaagtgaccatgagctgcaagagcagccagagcctgctgtactcccggaaccagaagaactacctggcctggttccagcagaagcccggccagtctcctaagctgctgatcttctgggccagcaccagagaaagcggcgtgcccgatagattcaccggcagcggctttggcaccgacttcaacctgacaatcagcagcgtgcaggccgaggacctggctgtgtacgattgccagcagtacttcagctaccctctgacctttggagccggcaccaagctggaactgagaggcggcggaggaagcggaggcggaggatctgggggcggaggctctggcggagggggatctcaggttcagctgcagcagtctggacctgagctggttaagcctggcgcctccgtgaagatgagctgcaagaccagccggtacaccttcaccgagtacaccatccactgggtccgacagagccacggcaagagcctggaatggatcggcggcatcaaccccaacaacggcatccccaactacaaccagaagttcaagggcagagccacactgaccgtgggcaagtctagcagcaccgcctacatggaactgcggagcctgacaagcgaggacagcgccgtgtacttctgcgccagaagaagaatcgcctacggctacgatgagggccacgccatggattattggggccagggaacaagcgtgaccgtgtctagt

SEQ ID NO: 94 - DNA, 840 bp, Artificial Sequence
  source: /note=“Description of Artificial Sequence: Synthetic polynucleotide”
atggactggatctggcggattctgtttctcgtgggagctgccacaggcgctcattctgctcagcctgcccaggttcagctgcagcagtctggacctgagctggttaagcctggcgcctccgtgaagatgagctgcaagaccagccggtacaccttcaccgagtacaccatccactgggtccgacagagccacggcaagagcctggaatggatcggcggcatcaaccccaacaacggcatccccaactacaaccagaagttcaagggcagagccacactgaccgtgggcaagtctagcagcaccgcctacatggaactgcggagcctgacaagcgaggacagcgccgtgtacttctgcgccagaagaagaatcgcctacggctacgatgagggccacgccatggattattggggccagggaacaagcgtgaccgtgtctagtggcggcggaggaagcggaggcggaggatctgggggcggaggctctggcggagggggatctgacatcgtgatgacacagagcccttctagcctggccgtgtccgtgggagagaaagtgaccatgagctgcaagagcagccagagcctgctgtactcccggaaccagaagaactacctggcctggttccagcagaagcccggccagtctcctaagctgctgatcttctgggccagcaccagagaaagcggcgtgcccgatagattcaccggcagcggctttggcaccgacttcaacctgacaatcagcagcgtgcaggccgaggacctggctgtgtacgattgccagcagtacttcagctaccctctgacctttggagccggcaccaagctggaactgaga

SEQ ID NO: 95 - protein, 280 aa, Artificial Sequence
  source: /note=“Description of Artificial Sequence: Synthetic polypeptide”
MDWIWRILFLVGAATGAHSAQPADIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELRGGGGSGGGGSGGGGSGGGGSQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSS

SEQ ID NO: 96 - protein, 280 aa, Artificial Sequence
  source: /note=“Description of Artificial Sequence: Synthetic polypeptide”
MDWIWRILFLVGAATGAHSAQPAQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSSGGGGSGGGGSGGGGSGGGGSDIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELR

SEQ ID NO: 97-112 - skipped (16 entries)

SEQ ID NO: 113 - DNA, 489 bp, Artificial Sequence
  source: /note=“Description of Artificial Sequence: Synthetic polynucleotide”
atgcgcatcagcaagccccacctgcgcagcatcagcatccagtgctacctgtgcctgctgctgaacagccacttcctgaccgaggccggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctaa

SEQ ID NO: 114 - protein, 162 aa, Artificial Sequence
  source: /note=“Description of Artificial Sequence: Synthetic polypeptide”
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 115 - protein, 4 aa, Unknown
  source: /note=“Description of Unknown: KDEL peptide”
KDEL

(19) United States
(12) Patent Application Publication
(10) Pub. No.: US 2026/0023069 A1
(43) Pub. Date: Jan. 22, 2026

(54) COMBINED INVASTION AND CYTOTOXICITY ASSAY USING CHEMOKINE SECRETING TARGET CELLS

(71) Applicant: ImmunityBio, Inc., San Diego, CA (US)

(72) Inventors: Nathan Thomas SCHOMER, San Diego, CA (US); Laurent H. BOISSEL, San Diego, CA (US); Hans G. KLINGEMANN, San Diego, CA (US)

(21) Appl. No.: 19/288,784

(22) Filed: Aug. 1, 2025

Related U.S. Application Data
(62) Division of application No. 17/264,601, filed on Jan. 29, 2021.
(60) Provisional application No. 62/713,287, filed on Aug. 1, 2018.

(51) Int. Cl. G01N 33/50; A61K 40/15; A61K 40/31; A61K 40/42

(52) U.S. Cl. CPC G01N 33/5029; A61K 40/15; A61K 40/31; A61K 40/4202; A61K 40/4211; A61K 40/4219; A61K 40/4234; G01N 33/5047; G01N 2333/521

(57) ABSTRACT

Provided herein are compositions and methods for detecting migration of effector cells towards a target cell, and cytotoxicity of the migrated effector cells against the target cells. The effector cells are modified to express a homing or migratory receptor, and the target cells are modified to express the cognate ligand. The methods can be carried out in a Boyden chamber or tranwells with a porous membrane between the wells. The membrane can be coated with an extracellular matrix component to simulate a solid tumor environment.

### RELATED APPLICATION

This application is a Division of U.S. application Ser. No. 17/264,601, filed Jan. 29, 2021, which is a U.S. National Stage Application under 35 U.S.C. § 371 of International Application No. PCT/US2019/044652, filed Aug. 1, 2019, which claims priority to U.S. Provisional Application No. 62/713,287, filed on Aug. 1, 2018. The entire contents is incorporated herein by reference for all purposes.

### SEQUENCE LISTING

The instant application contains a Sequence Listing which has been submitted electronically in ASCII format and is hereby incorporated by reference in its entirety. Said ASCII copy, created on Jul. 31, 2025, is named 104066-1518580_SEQ_LST_07_31_2025.txt and is 132,286 bytes in size.

### BACKGROUND

Cancer immunotherapies based on adoptively transferred tumor-specific cytotoxic lymphocytes hold promise for the treatment of patients with tumor malignancies. Despite this early success in certain cancers, the treatment of tumors remains a challenge, mostly due to the immunosuppressive nature of the tumor microenvironment. In addition to modified T-cells, immunotherapies based on NK cells are being explored. NK-92 is a cytolytic cancer cell line which was discovered in the blood of a subject suffering from a non-Hodgkin's lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells nor do they elicit an unacceptable immune rejection response in humans.

A common driver of lymph node metastasis is the hypoxia-driven upregulation of CCR7, a chemokine receptor primarily found in naïve T-cells and dendritic cells. Upregulation of the CCR7 receptor on blood NK cells has previously been demonstrated to improve the homing of NK cells to lymph nodes, allowing them to follow the same path to the lymph node compartments that are common pathways of metastatic spread, but has not yet been demonstrated in a clinically relevant cell line.

### BRIEF SUMMARY

Provided herein are methods for detecting migration and cytotoxicity of effector cells. The compositions and methods described herein provide the unexpected advantage of combining an effector cell that expresses a homing receptor with a target cell that expresses the cognate ligand (i.e., that binds to the receptor) in a single assay which measures both migration and cytotoxicity of effector cells. Another advantage of the methods described herein is the maintenance of a ligand gradient by the constant secretion of ligand from the target cells, so that the gradient does not degrade with time. These advantages represent a superior way to test the efficacy of cellular immunotherapeutics in a more clinically relevant scenario.

In one aspect, the method comprises:
providing a device comprising two or more chambers, wherein at least a first chamber is separated by a membrane from a second chamber, the membrane comprising pores having a diameter that permits an effector cell to pass through the membrane;
adding an effector cell to the first chamber, wherein the effector cell is modified to express a homing receptor;
adding a target cell to the second chamber, wherein the target cell is modified to express a ligand that binds to the homing receptor;
allowing the effector cell to migrate from the first chamber to the second chamber; and
measuring the number of target cells killed by the effector cells.

In some embodiments, the first chamber is located above the second chamber. In some embodiments, the membrane comprises an extra-cellular matrix component or analog that inhibits spontaneous migration of the effector cell.

In some embodiments, the effector cell is an NK-92 cell or a modified NK-92 cell described herein. In some embodiments, the effector cell is stained with a vital dye.

In some embodiments, the effector cell expresses a homing receptor, a cytokine receptor, or a chemokine receptor. In some embodiments, the homing receptor is one that is expressed in a tumor microenvironment. In some embodiments, the homing receptor is expressed by leukocytes, T or B lymphocytes, macrophages, natural killer cells, or dendritic cells. In some embodiments, the homing receptor is a chemokine receptor selected from CCR7 (SEQ ID NO: 14), CXCR2 (SEQ ID NO: 16), or the receptor for CXCL14 (SEQ ID NO: 10). In some embodiments, the homing receptor is CD62L (SEQ ID NO: 12). In some embodiments, the effector cell is a modified NK-92 cell.

In some embodiments, the target cell expresses a ligand that binds to a homing receptor (the cognate receptor-ligand pair). In some embodiments, the ligand is one that is expressed in a tumor microenvironment. In some embodiments, the ligand is expressed by tumor cells, lymphocytes, macrophages, natural killer cells, endothelial cells, or dendritic cells. In some embodiments, the ligand is a cytokine or chemokine. In some embodiments, the target cell expresses a chemokine that binds to the receptor. In some embodiments, the chemokine is CCL19 (SEQ ID NO: 2), CCL21 (SEQ ID NO: 4), interleukin 8 (IL8 or CXCL8 (SEQ ID NO: 6)), C-X-C motif chemokine ligand 1 (CXCL1 (SEQ ID NO: 8)), or C-X-C Motif Chemokine Ligand 14 (CXCL14 (SEQ ID NO: 10)). In some embodiments, the ligand binds CD62L (SEQ ID NO: 12).

In some embodiments, the effector cell further expresses a antigen binding protein or chimeric antigen receptor (CAR) that specifically binds a target antigen, such as a tumor associated antigen. In some embodiments, the tumor associated antigen is CD19, CD20, GD2, HER-2, CD30, EGFR, FAP, CD33, CD123, PD-L1, IGFIR, CSPG4, or B7-H4. In some embodiments, the effector cell expresses a CAR that specifically binds CD19 (SEQ ID NOs: 46, 47, and 63), CD20 (SEQ ID NO: 65), GD2 (SEQ ID NOs: 80 and 81), HER-2 (SEQ ID NO: 77), CD30 (SEQ ID NO: 75), EGFR (SEQ ID NO: 71), FAP (SEQ ID NOs: 95 and 96), CD33 (SEQ ID NO: 67), CD123 (SEQ ID NO: 86), PD-L1 (SEQ ID NO: 88), IGFIR (SEQ ID NO: 73), CSPG4 (SEQ ID NO: 69), or B7-H4 (SEQ ID NO: 90). In some embodiments, the CAR specifically binds: i) CD19 and comprises an amino acid sequence selected from SEQ ID NOs: 46, 47, or 63; ii) CD20 and comprises an amino acid sequence of SEQ ID NO: 65; iii) GD2 and comprises an amino acid sequence selected from SEQ ID NOs: 80 or 81; iv) HER-2 and comprises an amino acid sequence of SEQ ID NO: 77; v) CD30 and comprises an amino acid sequence of SEQ ID NO: 75; vi) EGFR and comprises an amino acid sequence of SEQ ID NO: 71; vii) FAP and comprises an amino acid sequence selected from SEQ ID NOs: 95 or 96; viii) CD33 and comprises an amino acid sequence of SEQ ID NO: 67; ix) CD123 and comprises an amino acid sequence of SEQ ID NO: 86; x) PD-L1 and comprises an amino acid sequence of SEQ ID NO: 88); xi) IGFIR and comprises an amino acid sequence of SEQ ID NO: 73; xii) CSPG4 and comprises an amino acid sequence of SEQ ID NO: 69; or xiii) B7-H4 and comprises an amino acid sequence of SEQ ID NO: 90.

In some embodiments, the target cell is a K562 cell or a HL-60 cell, or a modified K562 or HL-60 cell that expresses a ligand to the homing receptor, such as a cytokine or chemokine.

In some embodiments, the effector cell comprises a nucleic acid encoding a homing receptor operably linked to a promoter. In some embodiments, the effector cell comprises a nucleic acid encoding a CC chemokine receptor (CCR) or a CXC chemokine receptor (CXCR), such as CCR7 (SEQ ID NO: 13), CXCR2 (SEQ ID NO: 15), or the receptor for CXCL14 (SEQ ID NO: 9).

In some embodiments, the target cell comprises a nucleic acid encoding a ligand operably linked to a promoter. In some embodiments, the target cell comprises a nucleic acid encoding a chemokine operably linked to a promoter. In some embodiments, the target cell comprises a nucleic acid encoding the chemokine CCL19 (SEQ ID NO: 1), interleukin 8 (IL8 or CXCL8 (SEQ ID NO: 5)), C-X-C motif chemokine ligand 1 (CXCL1 (SEQ ID NO: 7)), or C-X-C Motif Chemokine Ligand 14 (CXCL14 (SEQ ID NO: 9)).

In another aspect, the modified effector cells express at least one cytokine. In some embodiments, the at least one cytokine is IL-2, IL-12, IL-15, IL-18, IL-21 or a variant thereof. In some embodiments, the at least one cytokine is IL-2, IL-15 or a combination thereof. In some embodiments, the IL-2 is expressed with a signal sequence that directs the IL-2 to the endoplasmic reticulum (referred to as “erIL-2” (SEQ ID NO: 25)). Directing the IL-2 to the endoplasmic reticulum permits expression of IL-2 at levels sufficient for autocrine activation and without releasing substantial amounts of IL-2 extracellularly. In some embodiments, the effector cells comprise a nucleic acid encoding a cytokine described herein. In some embodiments, the nucleic acid encodes a cytokine such as IL-2, IL-12, IL-15, IL-18, IL-21 or a variant thereof. In some embodiments, the nucleic acid encodes a cytokine such as erIL-2 (SEQ ID NO: 24) or erIL-15 (SEQ ID NO: 108).

In another aspect, the modified effector cells comprise a nucleic acid encoding an Fc receptor. In some embodiments, the Fc receptor is an Fc-gamma receptor (Cγr). In some embodiments, the Fc-gamma receptor is FCγRIII-A (also called CD16), which is a low affinity Fc receptor that binds to IgG antibodies and activates ADCC. In some embodiments, the CD16 receptor comprises a phenylalanine (F) to valine (V) substitution at amino acid position 158 (F158V) of the mature form of the polypeptide (corresponding to position 176 of the full length form of the polypeptide comprising the signal sequence). In one embodiment, the Fc receptor comprises the nucleic acid sequence of SEQ ID NO: 23 or the amino acid sequence of SEQ ID NO: 22.

In another aspect, the modified effector cells comprise a nucleic acid encoding an antigen binding protein (“ABP”). In some embodiments, the antigen binding protein specifically binds a tumor associated antigen. In some embodiments, the ABP comprises a fragment of an antibody, such as an scFv. In some embodiments, the antigen binding protein comprises or is part of a chimeric antigen receptor (CAR). In some embodiments, the nucleic acid encodes an ABP or CAR that specifically binds CD19 (SEQ ID NOs: 46, 47, and 63), CD20 (SEQ ID NO: 65), GD2 (SEQ ID NOs: 80 and 81), HER-2 (SEQ ID NO: 77), CD30 (SEQ ID NO: 75), EGFR (SEQ ID NO: 71), FAP (SEQ ID NOs: 95 and 96), CD33 (SEQ ID NO: 67), CD123 (SEQ ID NO: 86), PD-L1 (SEQ ID NO: 88), IGFIR (SEQ ID NO: 73), CSPG4 (SEQ ID NO: 69), or B7-H4 (SEQ ID NO: 90).

In another aspect, the modified effector cells express a cytokine or chemokine receptor, an ABP or CAR, an Fc receptor, and/or a cytokine. Thus, in some embodiments, the modified effector cells (e.g., NK-92 cells) express a chemokine receptor, a CAR, CD16, and erIL-2 (SEQ ID NO: 25). In some embodiments, the modified effector cells express CCR7 (SEQ ID NO: 14), a CAR that specifically binds CD19 (CD19-CAR (SEQ ID NOS: 46, 47, or 63), CD16 (SEQ ID NO: 22), and erIL-2 (SEQ ID NO: 25).

In some embodiments, the modified effector cells comprise one or more, or a plurality, of nucleic acid molecules encoding a cytokine or chemokine receptor, an ABP or CAR, an Fc receptor, and/or a cytokine. Thus, in some embodiments, the modified effector cells comprise nucleic acid molecules encoding a chemokine receptor, a CAR, CD16 (SEQ ID NO: 59), and erIL-2 (SEQ ID NO: 24). In some embodiments, the modified effector cells comprise nucleic acid molecules encoding CCR7 (SEQ ID NO: 13), a CAR that specifically binds CD19 (CD19-CAR (SEQ ID NO: 31 or 62)), CD16 (SEQ ID NOs: 23 or 59), and erIL-2 (SEQ ID NO: 24). In one embodiment, the nucleic acid molecule is RNA. In one embodiment, the nucleic acid molecule is DNA.

In some embodiments, the CAR comprises an intracellular signaling domain from the Fc epsilon receptor gamma (FcεRIγ (SEQ ID NO: 34)). In one embodiment, the CAR is transiently expressed by the effector cell. In one embodiment, the CAR is stably expressed by the effector cell.

In another aspect, an effector cell or cell line expressing a chimeric antigen receptor (CAR) on the surface of the effector cell is described, wherein said CAR comprises a cytoplasmic domain of FcεRIγ (SEQ ID NO: 34). In one embodiment, the cytoplasmic domain of FcεRIγ comprises an amino acid sequence having at least 95% sequence identity to SEQ ID NO: 34. In some embodiments, the cytoplasmic domain of FcεRIγ comprises or consists of the amino acid sequence of SEQ ID NO: 34.

In some embodiments, the cytoplasmic domain of FcεRIγ is encoded by a nucleic acid having at least 95% sequence identity to SEQ ID NO: 35.

In some embodiments, the CAR comprises a hinge region from CD8 (SEQ ID NO: 49). In some embodiments, the CAR comprises a transmembrane domain from CD28 (SEQ ID NO: 51).

In some embodiments, the effector cell or cell line is genetically modified with a nucleic acid construct that comprises SEQ ID NO: 35 (FcεRIγ), SEQ ID NO: 37 (CD8 hinge region), and/or SEQ ID NO: 51 (CD28 transmembrane domain). In some embodiments, the nucleic acid construct further comprises a promoter that promotes transcription of the nucleic acid sequences. In some embodiments, the promoter is an inducible promoter. In some embodiments, the nucleic acid construct is a multi-cistronic vector comprising one or more Internal Ribosome Entry Site (IRES (SEQ ID NO: 32)) to allow for initiation of translation from an internal region of an mRNA transcribed from the nucleic acid sequences. In some embodiments, the nucleic acid construct comprises a sequence that encodes a 2A peptide, such as a T2A (SEQ ID NO: 27), P2A (SEQ ID NO: 29), E2A (SEQ ID NO: 26 ), or F2A peptide, in order to produce equimolar levels of polypeptides encoded by the same mRNA. In some embodiments, the nucleic acid construct further comprises a nucleic acid sequence that encodes an antigen binding protein (ABP). In some embodiments, the ABP is an scFv or a codon optimized scFv. In some embodiments, the ABP specifically binds an antigen expressed by a tumor cell. In some embodiments, the ABP is part of a chimeric antigen receptor (CAR). In some embodiments, the construct comprises a nuclei acid that encodes a cytokine, such a IL-2. In one embodiment, the cytokine is targeted to the endoplasmic reticulum. In some embodiments, the construct comprises the vector shown in FIG. 5. In some embodiments, the NK-92 cell or cell line is genetically modified to express CD16 on the cell surface. In one embodiment, the NK-92 cell or cell line is genetically modified to express a high affinity CD16 (F158V) on the cell surface (SEQ ID NO: 23).

In one embodiment, the effector cell expresses an ABP or CAR that targets or specifically binds a tumor-associated antigen. In one embodiment, the tumor-associated antigen is selected from the group consisting of CD19, CD20, GD2, HER-2, CD30, EGFR, FAP, CD33, CD123, PD-L1, IGFIR, CSPG4, or B7-H4. In one embodiment, the tumor-associated antigen is CD19. In another embodiment, the tumor-associated antigen is CD33.

In one aspect, the present disclosure provides an effector cell line that is transformed by a nucleic acid encoding a chimeric antigen receptor (CAR) with a cytoplasmic domain of FcεRIγ (SEQ ID NO: 34), wherein the CAR is expressed on the surface of the NK-92 cell. In one embodiment, the nucleic acid is RNA. In one embodiment, the nucleic acid is DNA.

In some embodiments, the NK-92 cell is further modified to express at least one cytokine or variant thereof. In one embodiment, the at least one cytokine is transiently expressed by the NK-92 cell. In one embodiment, the at least one cytokine is stably expressed by the NK-92 cell. In some embodiments, the cytokine is IL-2, IL-15, or an IL-15 agonist, such as Altor-803.

In another aspect, the target cells comprise a nucleic acid encoding a chemokine that is expressed in lymph nodes. In some embodiments, the nucleic acid encoding the chemokine is expressed by an inducible promoter. In some embodiments, the target cells comprise a nucleic acid encoding the chemokine C-C motif ligand 21 (CCL21 (SEQ ID NO: 3)), a nucleic acid encoding the chemokine C-C motif ligand 19 (CCL19 (SEQ ID NO: 1)), or a combination thereof.

In some embodiments, the modified effector cells comprise an expression vector comprising one or more, or a plurality, of the nucleic acid molecules described herein. In some embodiments, the nucleic acid molecule is operably linked to a promoter that is capable of initiating transcription of the nucleic acid molecule. In some embodiments, each nucleic acid molecule of the plurality of nucleic acid molecules is operably linked to a separate, distinct and/or different promoter. In some embodiments, one or more of the nucleic acid molecules are operably linked to the same promoter. In one embodiment, the nucleic acid molecules encoding the cytokine or chemokine receptor, the CAR, the Fc receptor and the cytokine are operably linked to the same promoter or a single promoter. In some embodiments the promoter is an inducible promoter.

In some embodiments, the effector cells express the proteins encoded by the nucleic acid molecules described herein on the cell surface. For example, in some embodiments, the modified NK-92 cells express the cytokine/chemokine receptor, the ABP or CAR, and the Fc receptor (e.g., CD16 or high affinity CD16 (SEQ ID NO: 22)) on the cell surface.

Also provided are compositions and kits comprising the modified effector and target cells described herein. Also provided are methods of making the modified cells.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 is a schematic showing plasmid pNKAT-CCR7-LP3 containing the CCR7 receptor.

FIG. 2 shows graphs showing expression of phenotypic markers associated with NK-92 cells (CD54, NKp30, NKG2D) in wild type NK-92 cells and CCR7-expressing NK-92 cells clonal cells.

FIG. 3 is a graph showing cytoxic activity of modified NK-92 cells expressing CCR7 against K562 cells.

FIG. 4 is a graph showing cytoxic activity of modified NK-92 cells expressing CCR7 against HL-60 cells.

FIG. 5 shows a diagram representing an exemplary method for in vitro testing of the modified NK-92 cells described herein. Activated NK-92 cells (aNK) were modified to express a chemokine receptor (e.g., CCR7), and the target cells were modified to express a chemokine that binds to the receptor (e.g., CCL19 or CCL21). The modified NK-92 cells were tested in a Modified Boyden Chamber Transwell Assay as shown.

FIG. 6 is a graph showing modified NK-92 cells expressing CCR7 migrated towards the chemokines CCL19 and CCL21.

FIG. 7 shows a representative cytotoxicity assay using the modified NK-92 cells described herein. The modified NK-92 cells from FIG. 7 were tested for cytotoxicity against K562 target cells that express and secrete one or both chemokine ligands. The ML4 clone showed the highest percentage of lysis of target cells, and the percentage was increased when the K562 target cells expressed both CCL19 and CCL21.

FIG. 8 is a schematic showing plasmid pNKAT-CCR7-CD19CAR-CD16-ERIL2, referred to as a “Quadricistronic Vector,” which can be used to stably transfect a cell at a single insertion position.

FIG. 9 shows cell surface expression of CCR7, CD16, and CD19 CAR by NK-92 cells. “aNK” is the wild-type NK-92 cell line. “ML4” is the aNK cell line transfected with a nucleic acid construct encoding CCR7 operably linked to a promoter. “P2” is the aNK cell line transfected with a nucleic acid construct that encodes CCR7, CD16, ER-IL2 and CD19 CAR. The nucleic acids encoding i) CCR7, ii) CD16 plus ER-IL2, and iii) CD19 CAR were operably linked to the same promoter. “TC-CCR7aNK” is the aNK cell line transfected with a nucleic acid construct that encodes CCR7, CD16, ER-IL2 and CD19 CAR. The nucleic acids encoding i) CCR7 and ii) CD16 plus ER-IL2 plus CD19 CAR were operably linked to different promoters.

FIG. 10 shows a representative cytotoxicity assay using a modified NK-92 cell line (R7-19.1) transfected with a nucleic acid construct that encodes CCR7, CD16, ER-IL2 and CD19 CAR, compared to an unmodified NK-92 cell (aNK). Lysis of SUP-B15 target cells was greatly increased using the R7-19.1 cell line compared to unmodified NK-92 cells.

FIG. 11A and 11B show antibody dependent cellular cytotoxicity (ADCC) assays using the R7-19.1 cell line in combination with Herceptin (FIG. 11A) and Rituxumab (FIG. 11B) versus SUP-B15 (CD19−, CD20+) target cells.

### Definitions

Unless defined otherwise, all technical and scientific terms used herein have the same meaning as commonly understood by one of ordinary skill in the art in the field of immunology and immunotherapy. All references to a sequence identifier are understood to refer to exemplary sequences, and are not intended to exclude other sequences associated with a gene or protein molecule disclosed herein.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, include variations normally encountered by one of ordinary skill in the art. Therefore, numerical values can include variations of (+) or (−) increments of 0.1 or 1.0, where appropriate, depending on the relevant significant digit. It is to be understood, although not always explicitly stated, that all numerical designations may be preceded by the term “about.” The term “about” as used herein may also mean that the value can vary by ±1%, 2%, 3%, 4%, 5%, 6%, 7%, 8%, 9%, or 10%.

It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

As used herein, “immunotherapy” refers to the use of NK-92 cells, modified or unmodified, naturally occurring or modified NK cell or T-cell, whether alone or in combination, and which are capable of inducing cytotoxicity when contacting a target cell.

As used herein, “natural killer (NK) cells” are cells of the immune system that kill target cells in the absence of a specific antigenic stimulus, and without restriction according to major histocompatibility complex (MHC) class. NK cells are characterized by the presence of CD56 and the absence of CD3 surface markers.

The term “endogenous NK cells” is used to refer to NK cells derived from a donor (or the patient), as distinguished from the NK-92 cell line. Endogenous NK cells are generally heterogeneous populations of cells within which NK cells have been enriched. Endogenous NK cells may be intended for autologous or allogeneic treatment of a patient.

The term “NK-92” refers to natural killer cells derived from the highly potent unique cell line described in Gong et al. (1994), rights to which are owned by NantKwest (hereafter, “NK-92™ cells”). The immortal NK cell line was originally obtained from a patient having non-Hodgkin's lymphoma. Unless indicated otherwise, the term “NK-92™” is intended to refer to the original NK-92 cell lines as well as NK-92 cell lines that have been modified (e.g., by introduction of exogenous genes). NK-92™ cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92™, NK-92™-CD16, NK-92™-CD16-γ, NK-92™-CD16-ζ, NK-92™-CD16(F176V), NK-92™MI, and NK-92™CI. NK-92 cells are known to persons of ordinary skill in the art, to whom such cells are readily available from NantKwest, Inc.

The term “aNK” refers to unmodified natural killer cells derived from the highly potent unique cell line described in Gong et al. (1994), rights to which are owned by NantKwest (hereafter, “aNK™ cells”). The term “haNK” refers to natural killer cells derived from the highly potent unique cell line described in Gong et al. (1994), rights to which are owned by NantKwest, modified to express CD16 on the cell surface (hereafter, “CD16+NK-92™ cells” or “haNKR cells”). In some embodiments, the CD16+NK-92™ cells comprise a high affinity CD16 receptor on the cell surface. The term “taNK” refers to natural killer cells derived from the highly potent unique cell line described in Gong et al. (1994), rights to which are owned by NantKwest, modified to express a chimeric antigen receptor (hereafter, “CAR-modified NK-92™ cells” or “taNKR cells”). The term “t-haNK” refers to natural killer cells derived from the highly potent unique cell line described in Gong et al. (1994), rights to which are owned by Nantk West, modified to express CD 16 on the cell surface and to express a chimeric antigen receptor (hereafter, “CAR-modified CD16+NK-92™ cells” or “t-haNK™ cells”). In some embodiments, the t-haNK™ cells express a high affinity CD16 receptor on the cell surface.

The term “homing receptor” refers to a receptor that is involved in migration of a cell, such as an immune system cell or NK-92 cell, toward a specific target, such as tumor target tissue. The term is used interchangeably with “migratory receptor.”

The term “chemokine targeted t-haNK” refers to a t-haNK™ cell that is modified to express a chemokine receptor on the cell surface.

The term “Mi-aNK” refers to an aNK cell line transfected with a nucleic acid construct encoding a migratory receptor operably linked to a promoter. The term “Mi-T-haNK” refers to an aNK cell line transfected with a nucleic acid construct that encodes a migratory receptor, CD16, ER-IL2 and a CAR, each operably linked to the same or different promoters.

As used herein, the terms “cytotoxic” and “cytolytic,” when used to describe the activity of effector cells such as NK-92 cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK-92 cells is due to cytolysis.

The term “kill” with respect to a cell/cell population is directed to include any type of manipulation that will lead to the death of that cell/cell population.

The term “extracellular matrix analog” refers to a biocompatible material or hydrogel comprising one or more components of naturally occurring extracellular matrix (ECM). In some embodiments, the ECM analog is Corning® Matrigel® Matrix, which comprises solubilized basement membrane extracted from the Engelbreth-Holm-Swarm (EHS) mouse sarcoma, and includes ECM proteins such as laminin (a major component), collagen IV, heparin sulfate proteoglycans, entactin/nidogen, and a number of growth factors.

The term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified based on the type of antibody they recognize. For example, Fc-gamma receptors (FCγr) bind to the IgG class of antibodies. FCγRIII-A (also called CD16) is a low affinity Fc receptor that binds to IgG antibodies and activates ADCC. FCγRIII-A are typically found on NK cells. NK-92 cells do not express FCγRIII-A. Fc-epsilon receptors (FcεR) bind to the Fc region of IgE antibodies.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing NK-92 cell is targeted to cells expressing certain antigens on the cell surface, based on the specificity of the scFv domain. The scFv domain can be engineered to recognize any antigen, including tumor-specific antigens. For example, CD19CAR recognizes CD19, a cell surface marker expressed by some cancers.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

“Homology” or “identity” or “similarity” refers to sequence similarity between two peptides or between two nucleic acid molecules. Sequence similarity can be determined by comparing a position in each sequence which may be aligned for purposes of comparison. When a position in the compared sequence is occupied by the same base or amino acid, then the molecules are homologous at that position. The percent of sequence similarity between sequences is a function of the number of matching or homologous positions shared by the sequences over a given comparison window. A sequence can be at least 60%, 70%, 80%, 85%, 90%, 91%, 92%, 93%, 94%, 95%, 96%, 97%, 98% or 99% identical to a sequence described herein.

The terms identical or percent identity, in the context of two or more nucleic acids or polypeptide sequences, refer to two or more sequences or subsequences that are the same or have a specified percentage of amino acid residues or nucleotides that are the same (i.e., at least about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. In some embodiments, identity exists over a region that is at least about 25 amino acids or nucleotides in length, or over a region that is 50-100 amino acids or nucleotides in length.

For sequence comparison, typically one sequence acts as a reference sequence, to which test sequences are compared. When using a sequence comparison algorithm, test and reference sequences are entered into a computer; subsequence coordinates are designated, if necessary; and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150, in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, WI); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated for nucleotide sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4.

The term transformation as used herein refers to a process by which an exogenous or heterologous nucleic acid molecule (e.g., a vector or recombinant nucleic acid molecule) is introduced into a recipient cell. The exogenous or heterologous nucleic acid molecule may or may not be integrated into (i.e., covalently linked to) chromosomal DNA making up the genome of the host cell. For example, the exogenous or heterologous polynucleotide may be maintained on an episomal element, such as a plasmid. Alternatively or additionally, the exogenous or heterologous polynucleotide may become integrated into a chromosome so that it is inherited by daughter cells through chromosomal replication. Methods for transformation include, but are not limited to, calcium phosphate precipitation; fusion of recipient cells with bacterial protoplasts containing the recombinant nucleic acid; treatment of the recipient cells with liposomes containing the recombinant nucleic acid; DEAE dextran; fusion using polyethylene glycol (PEG); electroporation; magnetoporation; biolistic delivery; retroviral infection; lipofection; and micro-injection of DNA directly into cells.

The term transformed, as used in reference to cells, refers to cells that have undergone transformation as described herein such that the cells carry exogenous or heterologous genetic material (e.g., a recombinant nucleic acid). The term transformed can also or alternatively be used to refer to cells, types of cells, tissues, organisms, etc. that contain exogenous or heterologous genetic material.

The term introduce, as used herein with reference to introduction of a nucleic acid into a cell or organism, is intended to have its broadest meaning and to encompass introduction, for example by transformation methods (e.g., calcium-chloride-mediated transformation, electroporation, particle bombardment), and also introduction by other methods including transduction, conjugation, and mating. Optionally, a construct is utilized to introduce a nucleic acid into a cell or organism.

The terms modified and recombinant when used with reference to a cell, nucleic acid, polypeptide, vector, or the like indicates that the cell, nucleic acid, polypeptide, vector or the like has been modified by or is the result of laboratory methods and is non-naturally occurring. Thus, for example, modified cells include cells produced by or modified by laboratory methods, e.g., transformation methods for introducing nucleic acids into the cell. Modified cells can include nucleic acid sequences not found within the native (non-recombinant) form of the cells or can include nucleic acid sequences that have been altered, e.g., linked to a non-native promoter.

As used herein, the term exogenous refers to a substance, such as a nucleic acid (e.g., nucleic acids including regulatory sequences and/or genes) or polypeptide, that is artificially introduced into a cell or organism and/or does not naturally occur in the cell in which it is present. In other words, the substance, such as nucleic acid or polypeptide, originates from outside a cell or organism into which it is introduced. An exogenous nucleic acid can have a nucleotide sequence that is identical to that of a nucleic acid naturally present in the cell. For example, an NK-92 cell can be engineered to include a nucleic acid having a NK-92 sequence, e.g., heparanase. Optionally, an endogenous NK-92 heparanase sequence is operably linked to a gene with which the regulatory sequence is not involved under natural conditions. Although the NK-92 heparanase sequence may naturally occur in the host cell, the introduced nucleic acid is exogenous according to the present disclosure. An exogenous nucleic acid can have a nucleotide sequence that is different from that of any nucleic acid that is naturally present in the cell. For example, the exogenous nucleic acid can be a heterologous nucleic acid, i.e., a nucleic acid from a different species or organism. Thus, an exogenous nucleic acid can have a nucleic acid sequence that is identical to that of a nucleic acid that is naturally found in a source organism but that is different from the cell into which the exogenous nucleic acid is introduced. As used herein, the term endogenous, refers to a nucleic acid sequence that is native to a cell. As used herein, the term heterologous refers to a nucleic acid sequence that is not native to a cell, i.e., is from a different organism than the cell. The terms exogenous and endogenous or heterologous are not mutually exclusive. Thus, a nucleic acid sequence can be exogenous and endogenous, meaning the nucleic acid sequence can be introduced into a cell but have a sequence that is the same as or similar to the sequence of a nucleic acid naturally present in the cell. Similarly, a nucleic acid sequence can be exogenous and heterologous meaning the nucleic acid sequence can be introduced into a cell but have a sequence that is not native to the cell, e.g., a sequence from a different organism.

As described herein, a control or standard control refers to a sample, measurement, or value that serves as a reference, usually a known reference, for comparison to a test sample, measurement, or value. For example, a test cell, e.g., a cell transformed with nucleic acid sequences encoding genes for an Fc Receptor can be compared to a known normal (wild-type) cell (e.g., a standard control cell). A standard control can also represent an average measurement or value gathered from a population of cells (e.g., standard control cells) that do not express the Fc Receptor or that do not have or have minimal levels of Fc Receptor activity. One of skill will recognize that standard controls can be designed for assessment of any number of parameters (e.g., RNA levels, polypeptide levels, specific cell types, and the like).

The term “express” refers to the production of a gene product (e.g., a protein). The term “transient” when referring to expression means a polynucleotide is not incorporated into the genome of the cell. The term “stable” when referring to expression means a polynucleotide is incorporated into the genome of the cell, or a positive selection marker (i.e., an exogenous gene expressed by the cell that confers a benefit under certain growth conditions) is utilized to maintain expression of the transgene.

The term “cytokine” or “cytokines” refers to the general class of biological molecules which affect cells of the immune system. Exemplary cytokines include but are not limited to interferons and interleukins (IL)—in particular IL-2, IL-12, IL-15, IL-18 and IL-21. In preferred embodiments, the cytokine is IL-2.

As used herein, the term “vector” refers to a non-chromosomal nucleic acid comprising an intact replicon such that the vector may be replicated when placed within a permissive cell, for example by a process of transformation. A vector may replicate in one cell type, such as bacteria, but have limited or no ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In one embodiment, the vector is a viral vector, e.g. adenovirus. Viral vectors are well known in the art.

As used herein, the term “targeted,” when referring to protein expression, is intended to include, but is not limited to, directing proteins or polypeptides to appropriate destinations in the cell or outside of it. The targeting is typically achieved through signal peptides or targeting peptides, which are a stretch of amino acid residues in a polypeptide chain. These signal peptides can be located anywhere within a polypeptide sequence, but are often located on the N-terminus. Polypeptides can also be engineered to have a signal peptide on the C-terminus. Signal peptides can direct a polypeptide for extracellular section, location to plasma membrane, golgi, endosomes, endoplasmic reticulum, and other cellular compartments. For example, polypeptides with a particular amino acid sequence on their C-terminus (e.g., KDEL (SEQ ID NO: 115)) are retained in the ER lumen or transported back the ER lumen.

As used herein, the term “target,” when referring to targeting of a tumor, refers to the ability of NK-92 cells to recognize and kill a tumor cell (i.e., target cell). The term “targeted” in this context refers, for example, to the ability of a CAR expressed by the NK-92 cell to recognize and bind to a cell surface antigen expressed by the tumor.

As used herein, the term “transfect” refers to the insertion of nucleic acid into a cell. Transfection may be performed using any means that allows the nucleic acid to enter the cell. DNA and/or mRNA may be transfected into a cell. Preferably, a transfected cell expresses the gene product (i.e., protein) encoded by the nucleic acid.

Titles or subtitles may be used in the specification for the convenience of a reader, which are not intended to influence the scope of the present invention. Additionally, some terms used in this specification are more specifically defined below.

### DETAILED DESCRIPTION

Provided herein are compositions and methods for measuring the migratory or invasive potential of effector cells in the same assay as such cells are measured for cytotoxic capabilities following migration/invasion. In some embodiments, a modified target cell line (such as cancer cell line) is engineered to express a chemokine which it does not normally express, in order to establish a chemokine gradient in the assay which an effector cell with an appropriate receptor may recognize and use to migrate towards these target cells. Upon encountering the target cell, such a cell may be killed by the effector cell, and the number of such target cells which have been killed, or the difference in expected number of target cells by the end of the test, can be measured to provide a useful output in the form of a number expressing the relative number of targets killed in comparison to an appropriate control.

With the increasing attention being given to the possibilities of cellular immunotherapy, a robust assay for determining the capability of such therapeutic effector cells to both reach and then kill their targets (usually cancer cells in the patient) is of clear utility in improving the efficiency and ultimate success of such therapies. While hematological malignancies have demonstrated the high potential value of cellular immunotherapies, cellular immunotherapy has shown far less effectiveness in treating solid tumors. One theory for the decreased effectiveness in treating solid tumors is that the thick layer of extra-cellular matrix (ECM) provides a barrier to entry for effector cells to reach their targets. One way to measure the invasive potential of cells, and thus their ability to penetrate the ECM to reach their targets, is a Boyden Chamber Assay. In brief, this consists of two chambers separated by a membrane with pores having a diameter sufficient to allow effector cells to migrate between the two chambers. In some embodiments, the membrane comprises a layer of ECM components or an ECM analog (such as Matrigel) acting as an additional barrier to movement of effector cells. The effector cells are typically placed in the upper chamber, above the membrane and ECM analog, and the bottom well is filled with media containing a chemokine of interest, which acts as a chemoacttractant for effector cells that express the cognate chemokine receptor. The chambers are then left in a tissue culture incubator for some amount of time (for example 1 to 24 hours), and the upper chambers are then removed and the effector cells which have actively migrated through the ECM and the pores of the membrane now in the bottom well are counted. One of the disadvantages in the conventional approach is that only a single dose of chemokine is added to the bottom well at the beginning of the test, resulting in only a transient gradient in the Boyden Chamber.

Another common assay in the characterization of cellular immunotherapeutic agents is the cytotoxicity assay. While there are several variants of this assay, all involve co-incubating effector cells directly with target cells, waiting some amount of time, and then counting the number of target cells which have been killed. Usually, the effector to target ratio is varied to generate a range of values for the killing of the cells. If both migration/invasive capability and cytotoxic capability are being measured for the same effector cell line, this is usually done by means of two separate assays in isolation, and neither effect is dependent on the other, unlike in actual clinical applications.

Described herein is a method for combining these two assays, with an improvement in the behavior of the invasion assay due to maintenance of a constantly restored gradient, and a meaningful output summarizing the capability of an effector cell line to both invade through ECM based upon a chemokine gradient and then kill its target.

First, a target cell line which is susceptible to killing by the effector cell is selected, either due to natural recognition of target antigens or though CAR mediated or ADCC mediated killing of the target cells. This target cell is then engineered to constitutively express a chemokine of interest. For example, in the proof of concept embodiment described below, the target cell line is K562 (naturally susceptible to NK-mediated killing) and the chemokine of interest is CCL19 (the ligand for the CCR7 receptor). In order to produce a K562 cell line that expresses CCL19 constitutively, K562 cells are electroporated with a linearized plasmid (pNKAT-CONST-CCL19-LB) containing CCL19 cDNA driven by the EF1α promoter and a blasticidin selection cassette. The cells are then selected by exposure to Blasticidin, and the resulting polyclonal population is then subcloned by limited dilution cloning to get single-cell clonal populations of CCL 19 secreting K562 cells (here referred to as K-19 cells). Secretion of CCL19 was confirmed by ELISA.

Once an appropriate chemokine secreting cell line has been made, these target cells are added to the lower chamber of a Boyden Chamber assay to induce invasion, alongside appropriate controls (unmodified cells, no cells). A positive control may also consist of effector cells added directly into the lower chamber with the target cells to measure maximal killing with no required invasion. In some embodiments, the effector cells are stained with a vital dye, such as carboxyfluorescein succinimidyl ester (CFSE) or other long-term stain to differentiate them from target cells, and then placed in the upper chamber of the Boyden Chamber assay above the membrane and ECM analog. The combined assay components are then placed in a tissue culture incubator for an appropriate amount of time (overnight is typical, though with appropriate considerations longer assays of two or three days can also work).

After the incubation period, the chambers are removed from the incubator and the upper wells removed. Effector cells which have been drawn to the gradient will have migrated into the lower well, and target cells which are susceptible to killing by the effectors will show some level of induced lysis. In some embodiments, the number of dead cells can be measured on a flow cytometer by staining the cells with Propidium iodide (PI) or another live/dead cell staining dye to evaluate cell viability. The number of effector cells which have invaded the lower chamber can be counted by gating on the effector stain (for example, CFSE), and the degree of target elimination measured by comparing the number of target cells (non CFSE stained, in this example) which are positive for PI to the number which maintain membrane integrity to exclude PI. Note that in longer assays not all dead cells will be detectable by flow cytometry, as those killed early in the assay may no longer maintain the integrity to be identified as cells. The measure of effectiveness will relate to the difference in total target cell number compared to a control consisting of target cells by themselves and allowed to multiply without effector mediated killing.

The assay described herein provide a methodology for robustly examining the ability of prospective cellular immunotherapeutic agents to both reach their targets and eliminate them—an important diagnostic in measuring the clinical effectiveness of these therapeutic agents.

Also described herein are engineered cells using the cytotoxic activated Natural Killer cell line (NK-92 or aNK) as the basis to improve homing (migration) towards a target of interest. In some embodiments, the NK-92 cells are engineered to express a chemokine receptor (e.g., CCR7) known to direct lymphocytes to lymph nodes when expressed.

As described herein, modified effector cells have been generated with stable long-term expression of the CCR7 lymph node homing receptor driven by the Elongation Factor 1a (EF1a) promoter after electroporation with a linearized gene construct containing a CCR7 expression cassette along with a removable selection cassette comprising a selectable marker. After one week of Puromycin selection, followed by serial dilution cloning, monoclonal cell lines were established retaining a high level of CCR7 expression. These CCR7 overexpressing NK cells have functional responses to lymph node associated chemokines CCL21 and CCL19 in migration/invasion assays.

The NK-92 cell line is a human, IL-2-dependent NK cell line that was established from the peripheral blood mononuclear cells (PBMCs) of a 50-year-old male diagnosed with non-Hodgkin lymphoma (Gong, et al., Leukemia. 8:652-8 (1994)). NK-92 cells are characterized by the expression of CD56bright and CD2, in the absence of CD3, CD8, and CD16. A CD56bright/CD 16neg low phenotype is typical for a minor subset of NK cells in peripheral blood, which have immunomodulatory functions as cytokine producers. Unlike normal NK cells, NK-92 lacks expression of most killer cell inhibitor receptors (KIRs) (Maki, et al., J Hematother Stem Cell Res. 10:369-83 (2001)). Only KIR2DL4, a KIR receptor with activating function and inhibitory potential that is expressed by all NK cells, was detected on the surface of NK-92. KIR2DL4 is considered to mediate inhibitory effects through binding to the HLA allele G (Suck, Cancer Immunol. Immunother. 65 (4): 485-92 (2015)). The predominant pathway of cytotoxic killing of NK-92 cells is through the perforin/esterase pathway; NK-92 expresses high levels of perforin and granzyme B (Maki, et al., J Hematother Stem Cell Res. 10:369-83 (2001)).

NK-92 cells have a very broad cytotoxic range and are active against cell lines derived from hematologic malignancies and solid tumors (Klingemann, Blood, 87 (11): 4913-4 (1996); Swift, Haematologica. 97 (7): 1020-8 (2012); Yan, et al., Clin Cancer Res. 4:2859-68 (1998)). Safety assessments in severe combined immunodeficiency (SCID) mice showed no NK-92 treatment-related effects, such as acute toxicity or long-term carcinogenicity (Tam, et al., J Hematother. 8:281-90 (1999), Yan, et al., Clin Cancer Res. 4:2859-68 (1998)). Administration of NK-92 cells to mice challenged with human leukemia cells or mouse models of human melanoma resulted in improved survival and suppression of tumor growth, including complete remissions in some mouse tumors (Tam, et al., J Hematother. 8:281-90 (1999), Yan, et al., Clin Cancer Res. 4:2859-68 (1998)). Phase I clinical trials have confirmed its safety profile. Characterization of the NK-92 cell line is disclosed in WO 1998/49268 and U.S. Patent Application Publication No. 2002-0068044, which are incorporated by reference herein in their entireties.

Optionally, the modified effector cells may also express the Fc receptor CD16. As used herein, the term “Fc receptor” refers to a protein found on the surface of certain cells (e.g., natural killer cells) that contribute to the protective functions of the immune cells by binding to part of an antibody known as the Fc region. Binding of the Fc region of an antibody to the Fc receptor (FcR) of a cell stimulates phagocytic or cytotoxic activity of a cell via antibody-mediated phagocytosis or antibody-dependent cell-mediated cytotoxicity (ADCC). FcRs are classified by the type of antibody they recognize. For example, Fc-gamma receptors (FCγR) bind to the IgG class of antibodies. FCγRIII-A (also called CD16) is a low affinity Fc receptor that binds to IgG antibodies and activates ADCC. FCγRIII-A are typically found on NK cells. A representative amino acid sequence encoding CD16 is shown in SEQ ID NO: 22. A representative polynucleotide sequence encoding CD16 is shown in SEQ ID NO: 23. The complete sequences of CD16 can be found in the SwissProt database as entry P08637.

In some embodiments, the CD16 receptor comprises a phenylalanine (F) to valine (V) substitution at amino acid position 158 (F158V) in the IgG binding domain of the mature CD16 receptor (corresponding to Val at position 176 of the full length protein), which effects the antibody-dependent cell cytotoxic (ADCC) function of NK cells. The CD16 158V variant binds with higher affinity to human IgG1 and IgG3 than the 158F variant.

Optionally, the modified effector cells comprise a nucleic acid sequence with 70%, 80%, 90%, or 95% identity to SEQ ID NO: 23. Optionally, the modified effector cells comprise a nucleic acid sequence with 90%, 91%, 92%, 93%, 94%, 95%, 96%, 97%, 98% or 99% identity to SEQ ID NO: 23. Optionally, the modified effector cells comprise a polypeptide with 70%, 80%, 90%, or 95% identity to SEQ ID NO: 22 (having valine at position 176 of the full length polypeptide). Optionally, the modified effector cells comprise a polypeptide with 90%, 91%, 92%, 93%, 94%, 95%, 96%, 97%, 98% or 99% identity to SEQ ID NO: 22.

The cytotoxicity of NK-92 cells is dependent on the presence of cytokines (e.g., interleukin-2 (IL-2)). Thus, optionally, modified NK-92 cells are further modified to express at least one cytokine. Optionally, the at least one cytokine is IL-2, IL-12, IL-15, IL-18, IL-21 or a variant thereof. Optionally, the at least one cytokine is IL-2, IL-15 or a combination thereof. Optionally, the IL-2 or IL-15 are expressed with a signal sequence that directs the IL-2 or IL-15 to the endoplasmic reticulum (erIL-2 or erIL-15). Directing the IL-2 to the endoplasmic reticulum permits expression of IL-2 at levels sufficient for autocrine activation and without releasing substantial amounts of IL-2 extracellularly. See Konstantinidis et al “Targeting IL-2 to the endoplasmic reticulum confines autocrine growth stimulation to NK-92 cells” Exp Hematol. 2005 February; 33 (2):159-64. A representative nucleic acid encoding IL-2 is shown in SEQ ID NO: 24 and a representative polypeptide of IL-2 is shown in SEQ ID NO: 25.

Optionally, the modified effector cells comprise a nucleic acid sequence with 70%, 80%, 90%, or 95% identity to SEQ ID NO: 24. Optionally, the modified NK-92 cells comprise a nucleic acid sequence with 90%, 91%, 92%, 93%, 94%, 95%, 96%, 97%, 98% or 99% identity to SEQ ID NO: 24. Optionally, the modified NK-92 cells comprise a polypeptide with 70%, 80%, 90%, or 95% identity to SEQ ID NO: 25. Optionally, the modified NK-92 cells comprise a polypeptide with 90%, 91%, 92%, 93%, 94%, 95%, 96%, 97%, 98% or 99% identity to SEQ ID NO: 25. The provided modified NK-92 cells advantageously are capable of being maintained in the absence of IL-2 without secreting IL-2 in an amount to cause a clinical adverse effect.

### Chimeric Antigen Receptors

In some embodiments, the modified effector cells are further engineered to express a chimeric antigen receptor (CAR) on the cell surface. Optionally, the CAR is specific for a tumor-specific antigen. Tumor-specific antigens are described, by way of non-limiting example, in US 2013/0189268; WO 1999024566 A1; U.S. Pat. No. 7,098,008; and WO 2000020460 A1, each of which is incorporated herein by reference in its entirety. Tumor-specific antigens include, without limitation, CD19, CD20, GD2, HER-2, CD30, EGFR, FAP, CD33, CD123, PD-L1, IGFIR, CSPG4, or B7-H4. CARs can be engineered as described, for example, in Patent Publication Nos. WO 2014039523; US 20140242701; US 20140274909; U.S. 20130280285; and WO 2014099671, each of which is incorporated herein by reference in its entirety. Optionally, the CAR is a CD19 CAR, a CD33 CAR or CSPG-4 CAR.

### Homing Receptors

Provided herein are modified effector cells expressing a homing receptor (also referred to as a migratory receptor). In some embodiments, the homing receptor is a cytokine receptor or chemokine receptor. In some embodiments, the chemokine receptor is C-C chemokine receptor type 7 (CCR7), CXCR2, or a receptor for CXCL14. In some embodiments, the modified effector cells comprise a nucleic acid encoding a homing, cytokine or chemokine receptor. In some embodiments, the nucleic acid encoding the homing, cytokine or chemokine receptor is operably linked to a promoter. In some embodiments, the nucleic acid encodes C-C chemokine receptor type 7 (CCR7), CXCR2, or a receptor for CXCL14, and is operably linked to a promoter. In some embodiments, the nucleic acid encoding CCR7 has at least 90%, 91%, 92%, 93%, 94%, 95%, 96%, 97%, 98% or 99% identity to SEQ ID NO: 13. In some embodiments, the nucleic acid encoding CXCR2 has at least 90%, 91%, 92%, 93%, 94%, 95%, 96%, 97%, 98% or 99% identity to SEQ ID NO: 15. In some embodiments, the nucleic acid encoding the receptor for CXCL14 has at least 90%, 91%, 92%, 93%, 94%, 95%, 96%, 97%, 98% or 99% identity to SEQ ID NO: 9. Optionally, the homing, cytokine or chemokine receptor is expressed on the cell surface of the modified NK-92 cells.

In some embodiments, the modified target cell expresses a ligand that binds to its cognate receptor expressed by the effector cell. In some embodiments, the ligand is a cytokine or chemokine that binds to a receptor expressed by the effector cell. In some embodiments, the ligand is expressed in the tumor microenvironment, or is expressed by cells that direct homing to lymph nodes. In some embodiments, the target cell comprises a nucleic acid encoding C-C motif ligand 21 (CCL21) operably linked to a promoter. Optionally, the nucleic acid encoding CCL21 has at least 90%, 91%, 92%, 93%, 94%, 95%, 96%, 97%, 98% or 99% identity to SEQ ID NO: 3. Optionally, the modified target cell comprises a nucleic acid encoding C-C motif ligand 19 (CCL19) operably linked to a promoter. Optionally, the nucleic acid encoding CCL 19 has at least 90%, 91%, 92%, 93%, 94%, 95%, 96%, 97%, 98% or 99% identity to SEQ ID NO: 1. Optionally, the modified target cells comprise a nucleic acid encoding CCL19 and CCL21. Optionally, the nucleic acid encoding CCL21 is linked to the nucleic acid encoding CCL19 by a 2A peptide linker. Optionally, the 2A peptide linker comprises SEQ ID NO: 26 or 27.

### Expression Vectors

Provided herein are expression vectors comprising one or more nucleic acid sequences operably linked to a promoter. The nucleic acid sequences encoding the different elements of the vector can each be operably linked to the same or different promoters. In some embodiments, the expression vector comprises a nucleic acid encoding a homing receptor, a cytokine receptor or chemokine receptor. In one embodiment, the nucleic acid encodes the chemokine receptor CCR7, CXCR2 or the receptor for CXCL14. In some embodiments, the expression vector comprises a nucleic acid encoding a cytokine or chemokine. In one embodiment, the nucleic acid encodes the chemokine CCL19, CCL21, IL-8, or CXCL14. Optionally, the nucleic acid encoding CCL21 and CCL19 are linked by a 2A peptide linker. Exemplary promoters include, but are not limited to, the CMV promoter, ubiquitin promoter, PGK promoter, and EF1 also promoter. Optionally, provided herein are expression vectors comprising a nucleic acid sequence of SEQ ID NO: 13 or a nucleic acid with 90%, 91%, 92%, 93%, 94%, 95%, 96%, 97%, 98% or 99% identity to SEQ ID NO: 13. Optionally, the nucleic acid is operably linked to a promoter. Optionally, the promoter is selected from the group consisting of SEQ ID NOs: 17, 19, 20 or 21 or a promoter having 90%, 91%, 92%, 93%, 94%, 95%, 96%, 97%, 98% or 99% identity to SEQ ID NOs: 17, 19, 20 or 21. Also provided are expression vectors comprising a nucleic acid sequence of SEQ ID NO: 3 or a nucleic acid with 90%, 91%, 92%, 93%, 94%, 95%, 96%, 97%, 98% or 99% identity to SEQ ID NO: 3. (CCL21) Optionally, the nucleic acid is operably linked to a promoter.

In some embodiments, the provided expression vector comprises SEQ ID NO: 13 or a nucleic acid with 90%, 91%, 92%, 93%, 94%, 95%, 96%, 97%, 98% or 99% identity to SEQ ID NO: 13; SEQ ID NOs: 31 or 62 (CD19 CAR or CD19 scFv) or a nucleic acid with 90%, 91%, 92%, 93%, 94%, 95%, 96%, 97%, 98% or 99% identity to SEQ ID NOs: 31 or 62; SEQ ID NO: 59 (CD16 158V), or a nucleic acid with 90%, 91%, 92%, 93%, 94%, 95%, 96%, 97%, 98% or 99% identity to SEQ ID NO: 59; and/or SEQ ID NO: 24 (erIL-2), or a nucleic acid with 90%, 91%, 92%, 93%, 94%, 95%, 96%, 97%, 98% or 99% identity to SEQ ID NO: 24. In some embodiments, the provided expression vector comprises SEQ ID NO: 15 (CXCR2) or a nucleic acid with 90%, 91%, 92%, 93%, 94%, 95%, 96%, 97%, 98% or 99% identity to SEQ ID NO: 15; SEQ ID NOs: 31 or 62 (CD19 CAR or CD19 scFv) or a nucleic acid with 90%, 91%, 92%, 93%, 94%, 95%, 96%, 97%, 98% or 99% identity to SEQ ID NOs: 31 or 62; and SEQ ID NO: 59 (CD16 158V), and/or a nucleic acid with 90%, 91%, 92%, 93%, 94%, 95%, 96%, 97%, 98% or 99% identity to SEQ ID NO: 59; and/or SEQ ID NO: 24 (erIL-2), or a nucleic acid with 90%, 91%, 92%, 93%, 94%, 95%, 96%, 97%, 98% or 99% identity to SEQ ID NO: 24. Suitable expression vectors are known in the art and can be used. Optionally, the expression vector is a plasmid.

### Methods of Making Modified NK-92 Cells

Provided herein are methods of making modified effector cells and target cells described herein. The methods include transforming effector or target cells with an expression vector comprising a nucleic acid described herein operably linked to a promoter.

As used herein, the terms promoter, promoter element, and regulatory sequence refer to a polynucleotide that regulates expression of a selected polynucleotide sequence operably linked to the promoter, and that effects expression of the selected polynucleotide sequence in cells. In some embodiments, a promoter element is or comprises untranslated regions (UTR) in a position 5′ of coding sequences. 5′ UTRs form part of the mRNA transcript and so are an integral part of protein expression in eukaryotic organisms. Following transcription 5′UTRs can regulate protein expression at both the transcription and translation levels. Promoters controlling transcription from vectors in mammalian host cells may be obtained from various sources, for example, the genomes of viruses such as polyoma, Simian Virus 40 (SV40), adenovirus, retroviruses, hepatitis B virus and cytomegalovirus (e.g., SEQ ID NO: 21), or from heterologous mammalian promoters, e.g. beta actin promoter, Eukaryotic translation elongation factor 1 alpha 1 (EF1α) promoter (e.g., SEQ ID NO: 17), phosphoglycerate kinase (PGK) promoter (e.g., SEQ ID NO: 20) and ubiquitin promoter (e.g., SEQ ID NO: 19). Provided herein are promoters having 90%, 91%, 92%, 93%, 94%, 95%, 96%, 97%, 98% or 99% identity to SEQ ID NOs: 17, 19, 20 or 21.

The phrase selectable marker, as used herein, refers either to a nucleotide sequence, e.g., a gene, that encodes a product (polypeptide) that allows for selection, or to the gene product (e.g., polypeptide) itself. The term selectable marker is used herein as it is generally understood in the art and refers to a marker whose presence within a cell or organism confers a significant growth or survival advantage or disadvantage on the cell or organism under certain defined culture conditions (selective conditions). The phrase selection agent, as used herein refers to an agent that introduces a selective pressure on a cell or populations of cells either in favor of or against the cell or population of cells that bear a selectable marker. For example, the selection agent is an antibiotic and the selectable marker is an antibiotic resistance gene. Examples of suitable selectable markers for mammalian cells are dihydrofolate reductase (DHFR), thymidine kinase, neomycin, neomycin analog G418, hydromycin, and puromycin.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

### Kits

Provided herein are kits comprising the modified effector and target cells described herein. In some embodiments, the kit comprises modified effector cells that express a homing receptor, a cytokine receptor or a chemokine receptor. In some embodiments, the kit comprises modified effector cells that also express a CAR described herein. In some embodiments, the kit comprises modified effector cells comprising one or more nucleic acid sequences encoding i) a homing, cytokine or chemokine receptor, ii) an ABP or CAR that specifically binds to a target antigen, iii) an Fc Receptor such as CD16 or CD16-158V, and/or iv) a cytokine such as erIL-2, operably linked to a promoter. In some embodiments, kit comprises a modified effecctor cell comprising a nucleic acid encoding C-C chemokine receptor type 7 (CCR7), CXCR2, or the receptor for CXCL14 operably linked to a promoter. Optionally, the nucleic acid encoding CCR7 has at least 90%, 91%, 92%, 93%, 94%, 95%, 96%, 97%, 98% or 99% identity to SEQ ID NO: 13. Optionally, the chemokine receptor is expressed on the cell surface of the modified effector cells.

In some embodiments, the kit comprises modified target cells that express a ligand (for example, a cytokine or chemokine that binds to its cognate receptor) expressed by the effector cells. In some embodiments, the modified target cell comprises a nucleic acid encoding C-C motif ligand 21 (CCL21) operably linked to a promoter. Optionally, the nucleic acid encoding CCL21 has at least 90%, 91%, 92%, 93%, 94%, 95%, 96%, 97%, 98% or 99% identity to SEQ ID NO: 3. In some embodiments, the modified target cell comprises a nucleic acid encoding C-C motif ligand 19 (CCL19) operably linked to a promoter. Optionally, the nucleic acid encoding CCL 19 has at least 90%, 91%, 92%, 93%, 94%, 95%, 96%, 97%, 98% or 99% identity to SEQ ID NO: 1. Optionally, the promoter drives expression of both CCL21 and CCL19. Optionally, the nucleic acid encoding CCL19 and CCL21 are linked by a 2A peptide linker, for example, SEQ ID NO: 26 or 27. In some embodiments, the kit includes instructions for using the kit components in the methods described herein. The instructions may further contain information regarding how to prepare (e.g., dilute or reconstitute, in the case of freeze-dried protein) the modified effector and target cells (e.g., thawing and/or culturing).

In some embodiments, the kit comprises one or more transwells. In some embodiments, the transwell comprises a porous membrane between the wells. In some embodiments, the membrane comprises an extracellular matrix component or analog that inhibits migration of the effector cell.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed while, specific references to each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1. Modified NK Cell Line Expressing CCR7

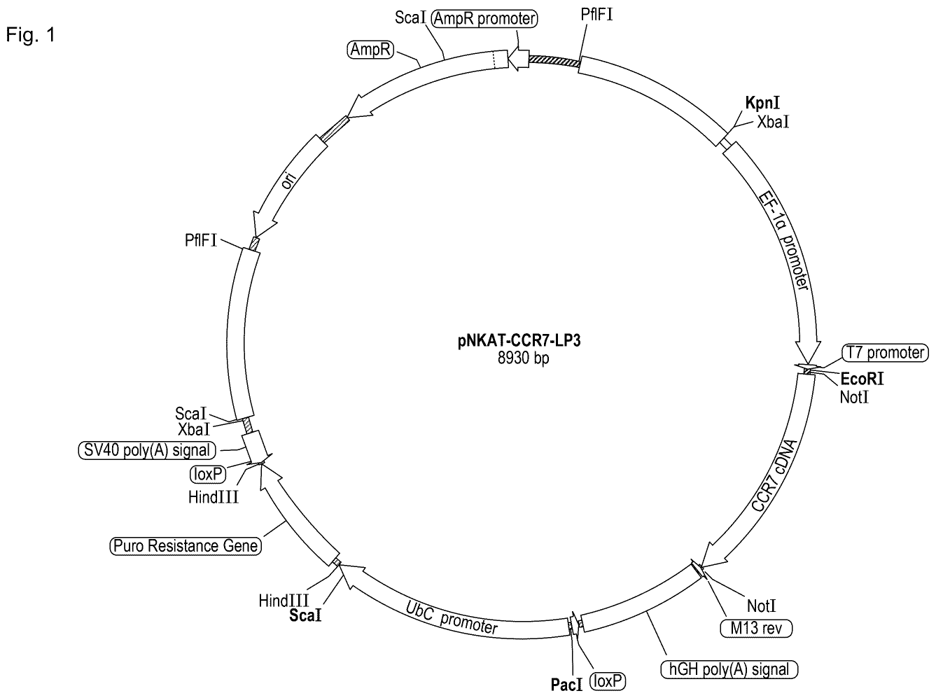

Modified NK-92 cells were made by electroporation with a linearized pNKAT-CCR7-LP3 plasmid (FIG. 1) using a NEON™ transfection system. After 1 week of puromycin selection, the resulting polyclonal population was tested for CCR7 expression, and monoclonal cell lines were derived by serial dilution in growth media supplemented with 5% human serum and IL-2. The modified NK-92 cells contained the EF1a promoter, CCR7 Gene with Poly-A tail, and the LoxP flanked puromycin resistance gene driven by the ubiquitin promoter (SEQ ID NO: 19).

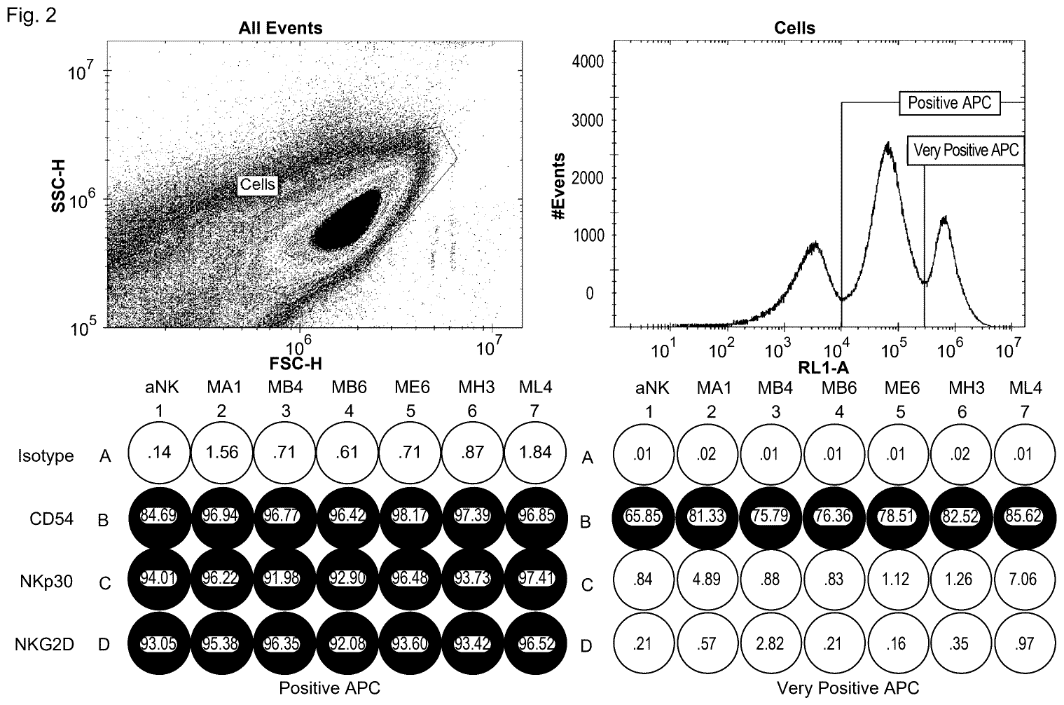

To verify expression of CCR7 does not affect NK-92 cells, expression of NK-92 cell markers was determined. The results are shown in FIG. 2. The data in FIG. 2 was generated using an Intellicyt iQue screener plus. Cells were incubated at 4° C. for 30 minutes with either APC conjugated antibody against the described phenotypic marker, or appropriate isotype as negative control. Cells were then rinsed in PBS+1% BSA, pelleted, and re-suspended in 30 uL of PBS+1% BSA. The readout was then gated as shown in the upper left quadrant to eliminate cellular debris from the readings, and the percentage of cells above the fluorescence thresholds shown in the upper right quadrant were then displayed as two separate heatmaps, showing the percentage above the “positive” threshold, and the “very positive” threshold in the lower left and lower right quadrants respectively. FIG. 2 shows that driving expression of CCR7 does not meaningfully affect the primary phenotypic markers associated with our cell lines. Specifically, CCR7 expression does not appear to affect CD54, NKp30 or NKG2D expression.

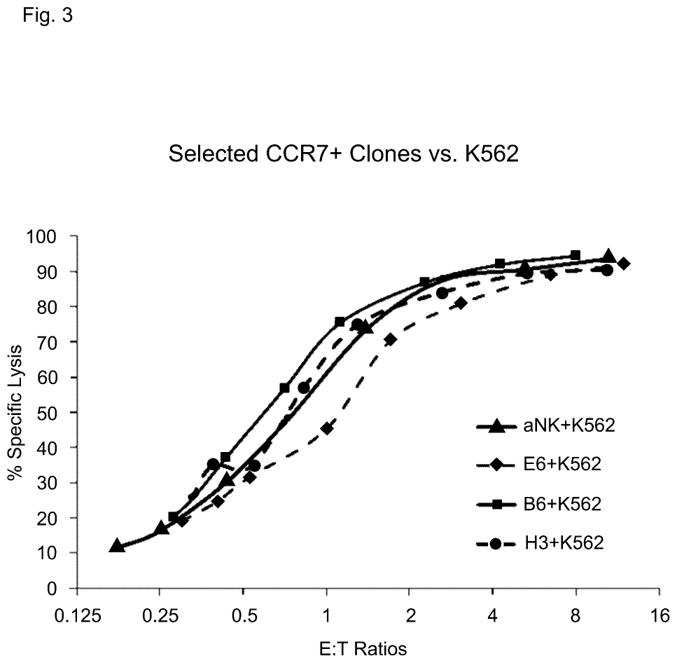

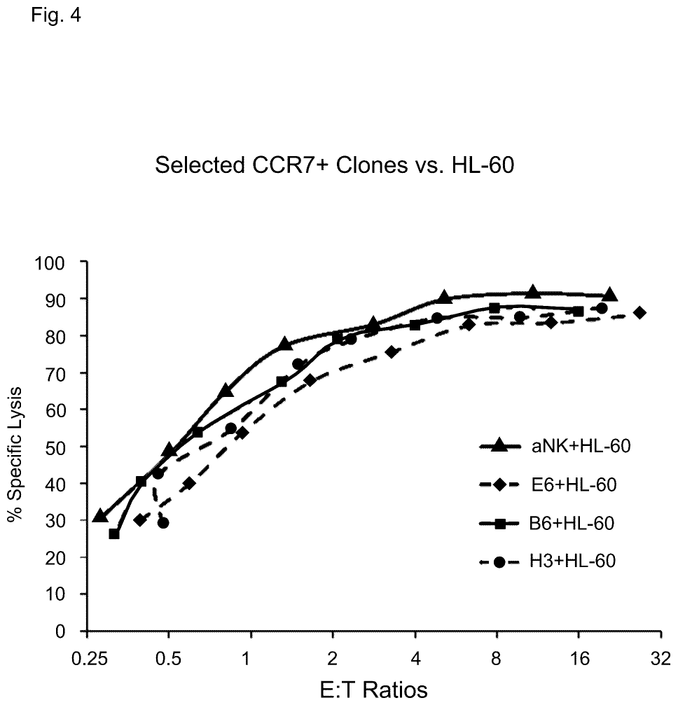

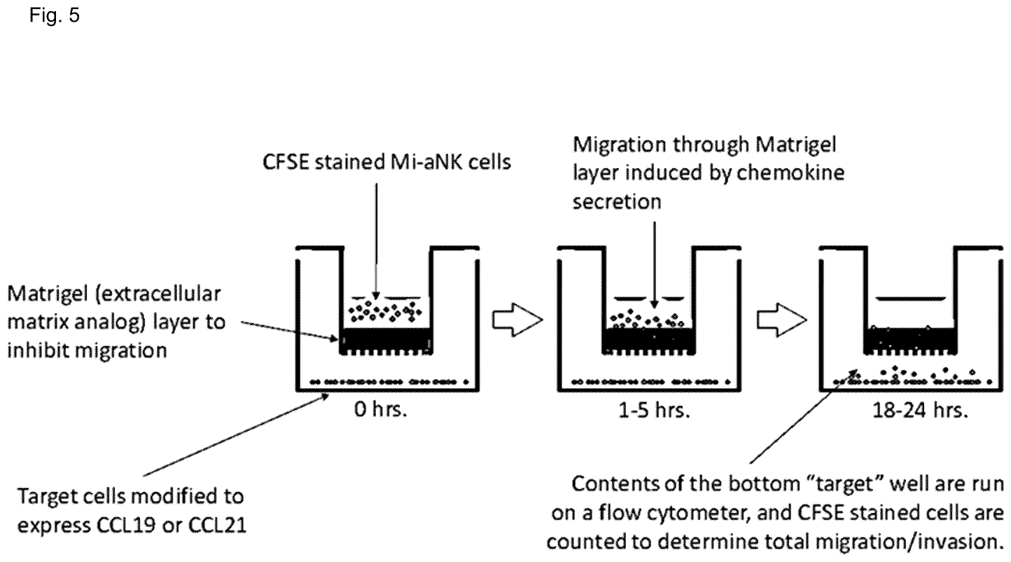

To determine cytotoxicity of the modified NK-92 cells, effector cells (NK-92 cells and modified NK-92 cell clones) were seeded into the left wells of a 96-well V-bottom plate (turned sideways), and then serially diluted across the plate, with 100k effectors left in the highest concentration well, and with 7 further 2-fold dilutions across the 8 rows of the plate. Stained target cells (K562 (FIG. 4), HL-60 (FIG. 5) were then seeded at 10k/well in all wells containing effectors, along with control wells of just targets to measure background death. The plate was then briefly spun down and incubated at 37° C. and 5% CO2 for 4 hours. The plate was then spun down, the supernatant aspirated off, and the cells re-suspended in PBS containing propidium iodide to measure cell death. The cells were then run through an Intellicyt iQue screener plus, and the proportion of target cells (differentiated from effectors by their stain) which are also positive for PI staining was measured. The percentage of dead cells was then compared against the number of naturally dying cells in the control wells, and a percentage of cells that are specifically killed by the effectors was calculated. The results are shown in FIGS. 3 and 4. FIG. 3 shows comparable cytotoxicity in CCR7 upregulated clones as compared to parental cell line vs. K562 cells and FIG. 4 shows comparable cytotoxicity in CCR7 upregulated clones as compared to parental cell line vs. HL-60 cells.

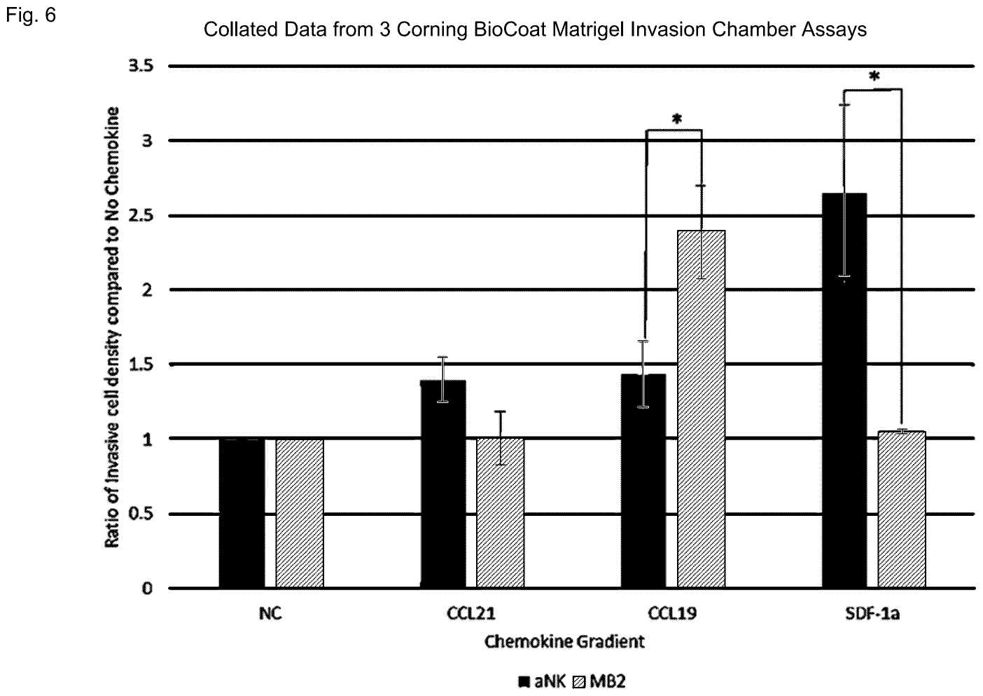

In vitro testing consisted of using Boyden chamber assays and a Matrigel layer to block migration. The modified cells expressing CCR7 showed migration towards CCL21 and CCL19 (an alternate CCR7 ligand) in these assays. Cells were placed in an upper well, and separated from a lower chamber by a thin layer of Matrigel (an ECM-like substrate) coated on 8 uM pores. The cells (25k/well) were placed in the upper chamber, in reduced-serum media (supplemented with 1% Human Serum+500U/mL IL-2), and the same reduced serum medium was used in the lower chamber, either by itself or containing a chemokine of interest. In this case, CCL21 was used at 15 ng/ml, CCL19 was used at 15 ng/ml, and SDF-1a was used at 20 ng/mL. Each test was done in triplicate for either NK-92 cells or modified NK-92 cells expressing CCR7. The plate was then placed in the incubator overnight for an 18 hour invasion assay, after which the upper chambers were removed, and 150 μL (of 750 μL total volume) was sampled from the lower well after thorough mixing and read on a MacsQuant FACS analysis machine. Live cells in the lower chamber were counted, and the number of cells was then compared against the wells containing no chemokine and an invasiveness index number was generated. These numbers were averaged and statistical relevance calculated using a two-tailed t-test. As the lower well was sampled without any detachment of cells from the lower membrane, those cells still attached to the lower portion of the ECM would not be represented in these numbers, likely resulting in the differences between CCL19 and CCL21. The results are shown in FIG. 6. Specifically, FIG. 6 shows statistically significant increases in invasiveness of modified NK-92 cells expressing CCR7 towards CCL19, a CCR7 chemokine. The lack of a statistically significant response to CCL21 is likely due to the nature of the assay performed. The assay measures both invasion and subsequent detachment from the ECM, a behavior consistent with CCL19 gradient migration. CCL21, while inducing migration, does not induce detachment from the matrix, requiring an additional step to demonstrate statistically significant invasive potential.

### Example 4: In Vitro Testing of Modified NK Cell Line Expressing CCR7

FIG. 5 shows the experimental protocol for in vitro testing of modified NK-92 cells transfected with nucleic acid constructs that express CCR7.

### Example 5: In Vitro Cytotoxicity Assays Using Modified NK Cell Line Expressing CCR7

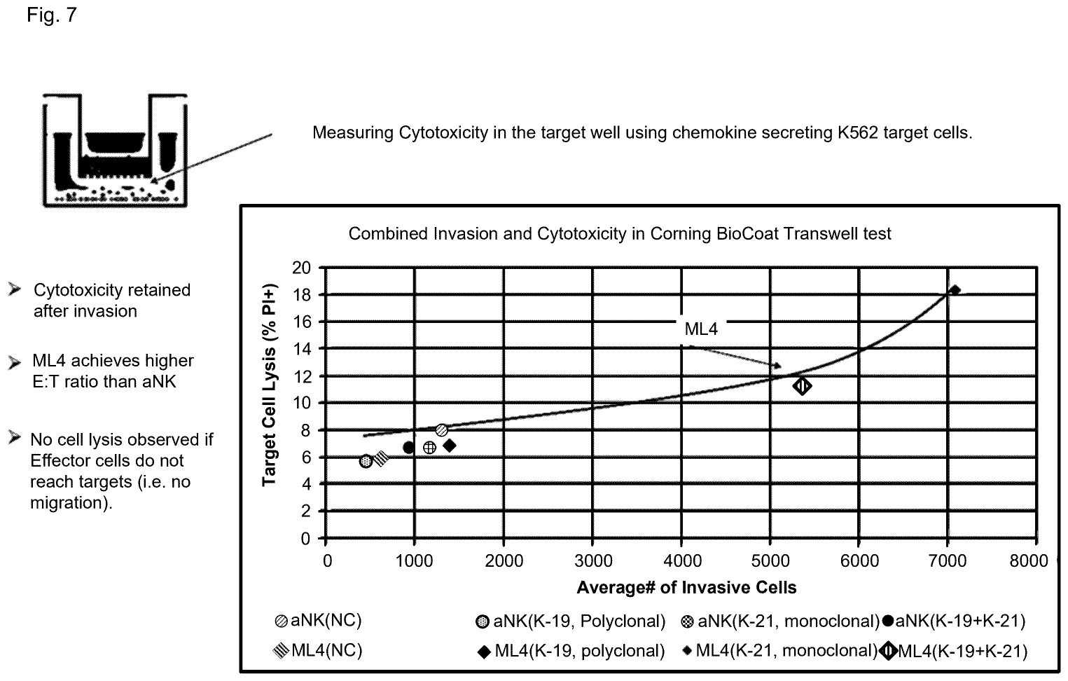

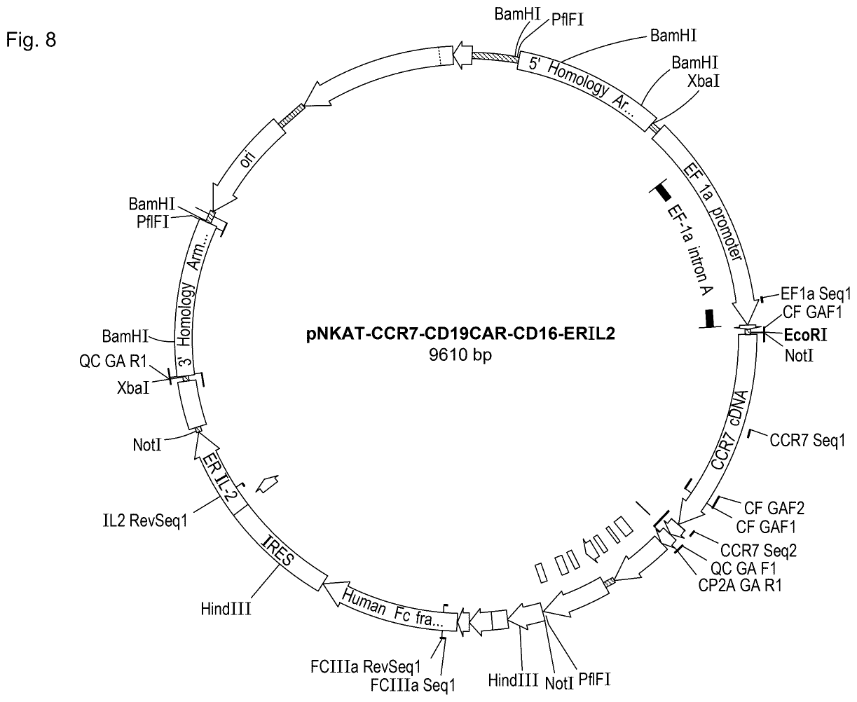

FIG. 7 shows that NK-92 cells modified to express CCR7 maintain cytotoxicity to target cells after migration in the modified Boyden chamber transwell assay as described in Example 1.

### Example 6: Cell Surface Expression of CCR7, CD16, and CD19 Car in NK-92 Cells Transfected With Nucleic Acid Constructs

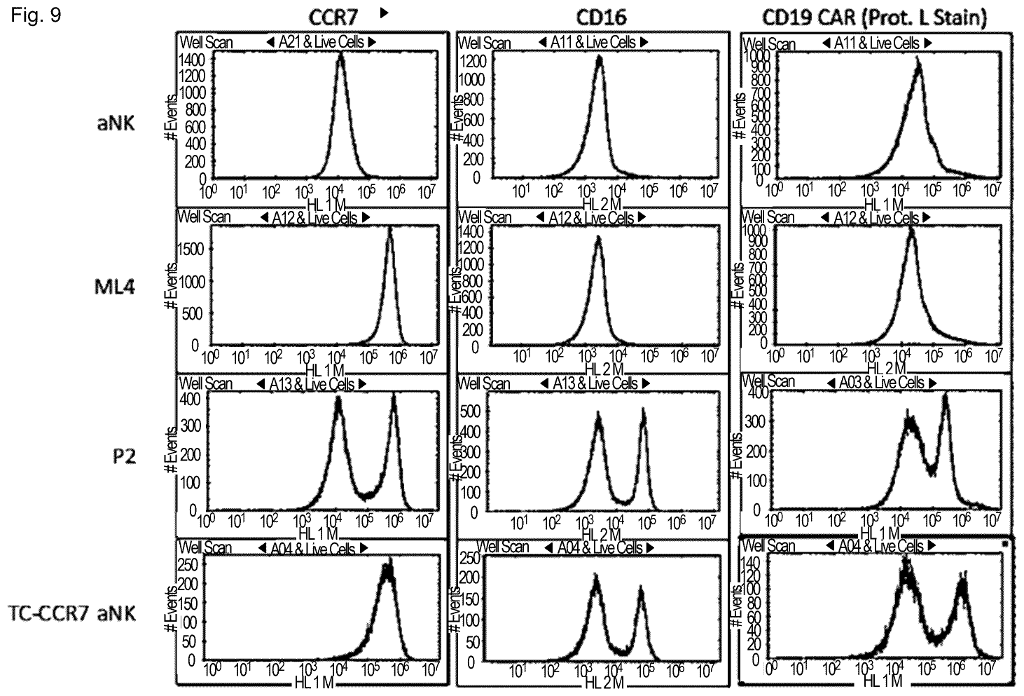

FIG. 9 shows that modified NK-92 cells transfected with nucleic acid constructs encoding CCR7, CD16, and CD19 CAR express high levels of the respective proteins on the cell surface.

### Example 7: Lysis of Target Cells By NK-92 Cells That Express CCR7, CD16, ER-IL2 AND CD19 CAR

This Example demonstrates that modified NK-92 cells transfected with nucleic acid constructs encoding CCR7, CD16, ER-IL2 and CD19 CAR efficiently lyse target cells compared to unmodified NK-92, and increase antibody dependent cellular cytotoxicity (ADCC) compared to control cells.

To determine cytotoxicity of the modified NK-92 cells (co-expressing CCR7, CD19 CAR, CD16, and ERIL-2), effector cells (aNK cells and R7-19.1 cell clone) were serially diluted across the plate, with 100k effectors left in the highest concentration well, and with 7 further 2-fold dilutions across the 8 rows of the plate. CFSE-stained target cells (SUP-B15, or SUP-B15 CD19−/CD20+) were then seeded at 10k/well in all wells containing effectors, along with control wells of just targets to measure background death. The plate was then briefly spun down and incubated at 37° C. and 5% CO2 for 4 hours. The plate was then centrifuged, the supernatant aspirated off, and the cells re-suspended in PBS containing propidium iodide to measure cell death. The cells were then run through an Intellicyt iQue screener plus, and the proportion of target cells (differentiated from effectors by their stain) which are also positive for PI staining was measured. The percentage of dead cells was then compared against the number of naturally dying cells in the control wells, and a percentage of cells that are specifically killed by the effectors was calculated. To determine ADCC, CFSE-stained SUP-B15 CD19−/CD20+ target cells were first incubated with rituxan antibody (anti-CD20) or Herceptin antibody (control, anti-HER2/neu) for 20 min before beige seeded in the plate containing effectors.

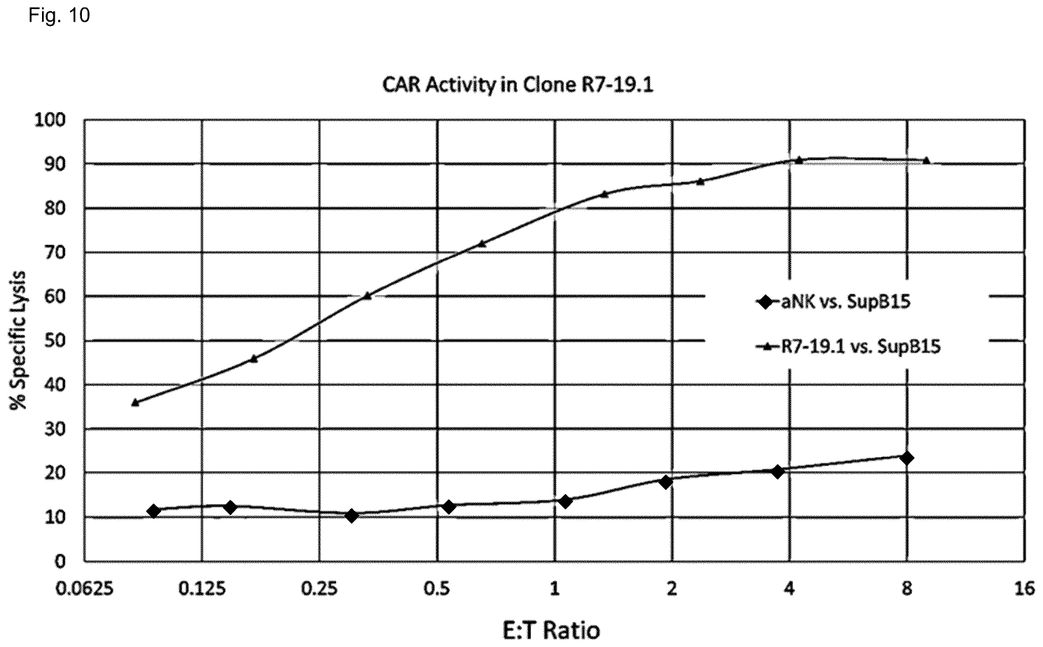

FIG. 10 shows a representative cytotoxicity assay using a modified NK-92 cell line (R7-19.1) transfected with a nucleic acid construct that encodes CCR7, CD16, ER-IL2 and CD19 CAR, compared to an unmodified NK-92 cell (aNK). Lysis of the CD19+, NK-resistant SUP-B15 target cells was greatly increased using the R7-19.1 cell line compared to unmodified NK-92 cells.

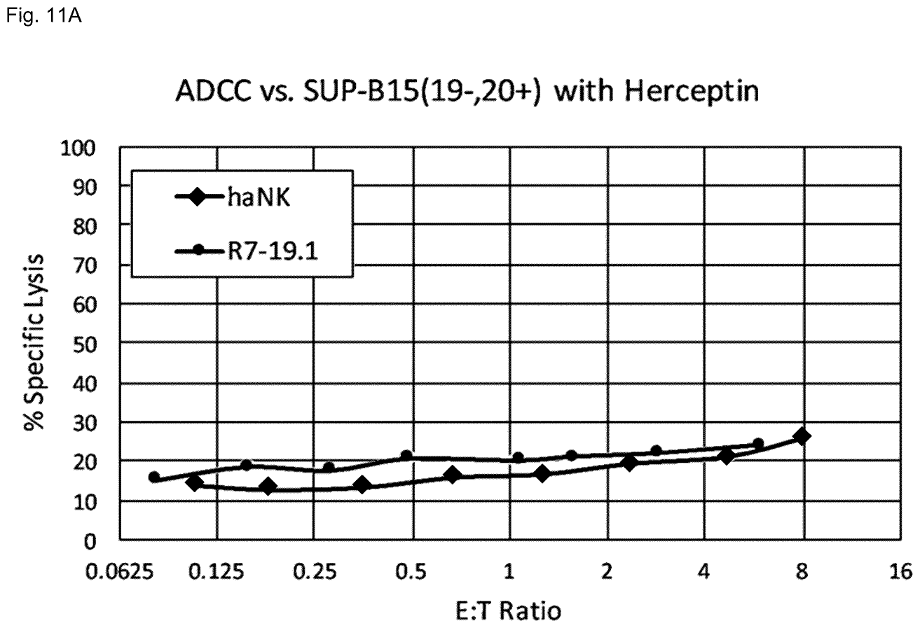

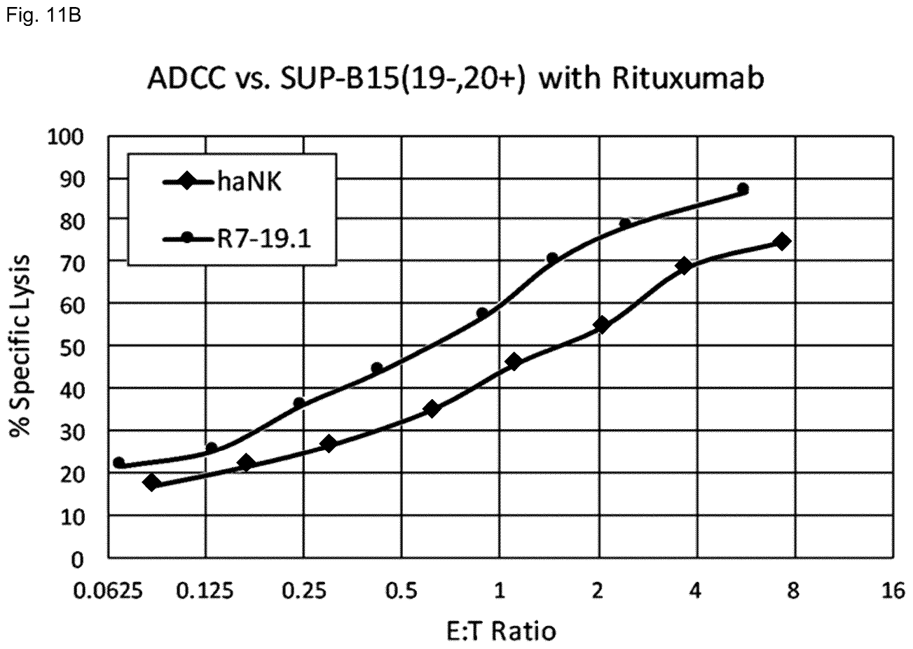

FIG. 11A and 11B show ADCC assays using the modified R7-19.1 NK-92 cell line in combination with Herceptin (FIG. 11A) and Rituxumab (FIG. 11B). The target cells are a variant of SUP-B15 cells that do not express CD19 (CD19−), but do express CD20 (CD20+). As can be seen in FIG. 11A, no increase in ADCC was observed with Herceptin compared to control haNK cells that express CD16. In contrast, there was an increase in ADCC with Rituxumab, which binds CD20, for both the R7-19.1 cell line and control haNK cells, and the R7-19.1 cell line showed increased cytotoxicity compared to control cells.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

What is claimed is:

1. A kit comprising a modified effector cell that expresses a cytokine receptor or chemokine receptor, and a modified target cell that expresses a cytokine or chemokine that binds to the receptor expressed by the effector cell.

2. The kit of claim 1, further comprising a transwell with or without an extracellular matrix coating.
